(19) United States
(12) Patent Application Publication
(10) Pub. No.: US 2004/0013643 A1
(43) Pub. Date: Jan. 22, 2004

(54) Methods for treatment of multiple sclerosis with statins

(75) Inventor: Francois Mach, Vesenaz (CH)

Correspondence Address: MINTZ, LEVIN, COHN, FERRIS, GLOVSKY, AND POPEO, P.C., ONE FINANCIAL CENTER, BOSTON, MA, 02111, US

(73) Assignee: Novlmmune S.A.

(21) Appl. No.: 10/349,549

(22) Filed: Jan. 22, 2003

Related U.S. Application Data
(63) Continuation-in-part of application No. 09/664,873, filed on Sep. 19, 2000, now Pat. No. 6,611,093.
(63) Continuation-in-part of application No. 09/960,471, filed on Sep. 19, 2001.
(63) Continuation-in-part of application No. 10/056,608, filed on Jan. 23, 2002.

(51) Int. Cl. A61K 38/21; A61K 31/65; A61K 31/59; A61K 31/505; A61K 31/401; A61K 31/366; A61K 31/557; A61K 31/35

(52) U.S. Cl. 424/85.6; 514/256; 514/423; 514/460; 514/548; 424/85.7; 514/153; 514/167; 514/573; 514/12; 514/492

(57) ABSTRACT

New uses of statins as novel types of immunomodulator. More specifically, the invention relates to methods for treating multiple sclerosis through the administration of one or more statins, and even more advantageously, in combination with other multiple sclerosis agents or treatments, such as β-interferons or copaxone.

### RELATED APPLICATIONS

This application is a continuation-in-part of copending U.S. application Ser. No. 09/960,471 filed September 19, 2001 which is a continuation-in-part of copending U.S. application Ser. No. 09/664,871 filed Sep. 19, 2000; and copending U.S. application Ser. No. 10/056,608 filed Jan. 23, 2002, the entire contents of all of which are incorporated herein by reference.

### FIELD OF THE INVENTION

The invention relates to a new use of statins as a novel type of immunomodulator. More specifically, the invention relates to methods for treating multiple sclerosis through the administration of one or more statins, and even more advantageously, in combination with other multiple sclerosis agents or treatments, such as β-interferons or copaxone.

### BACKGROUND OF THE INVENTION

The immune system is highly complex and tightly regulated, with many alternative pathways capable of compensating deficiencies in other parts of the system. There are occasions when the immune response becomes a cause of disease or other undesirable conditions, if activated. Such diseases or undesirable conditions include autoimmune diseases. The pathways leading to these undesired immune responses characteristic of autoimmune diseases such as multiple sclerosis (MS) often involve a common step—activation of lymphocytes.

The autoimmune disease multiple sclerosis is a progressive central nervous system (CNS) disease in which patches of myelin, the protective covering of nerve fibers, in the brain and spinal cord are destroyed by the body's own immune system via a chronic inflammatory autoimmune reaction. This destruction leads to scarring and damage to the underlying nerve fibers, and may manifest itself in a variety of symptoms, depending on the parts of the brain and spinal cord that are affected. Spinal cord damage may result in tingling or numbness, as well as a heavy and/or weak feeling in the extremities. Damage in the brain may result in muscle weakness, fatigue, unsteady gain, numbness, slurred speech, impaired vision, vertigo and the like.

In the animal model experimental autoimmune encephalomyelitis (EAE), immunizing susceptible rodent strains with CNS proteins such as myelin basic protein (MBP) induces an MS-like paralytic disease. Inflamed MS and EAE lesions, but not normal white matter, have infiltrating CD4 T-cells that respond to self antigens presented by MHC class II molecules like human HLA-DR2 (MS) or murine I-A^{u} (EAE). The infiltrating CD4 T-cells (Th1 cells) produce pro-inflammatory cytokines (interleukin (IL-2), interferon (IFN-γ), and tumor necrosis factor (TNF)-α) that activate antigen-presenting cells like macrophage to produce inflammatory cytokines (IL-1β, IL-6, and L-8) and IL-12. The L-12 induces further IFN-γ synthesis. In this cyclical manner, a chronic autoantigen-driven immune reaction is thought to produce a demyelinating attack on the CNS.

Several general therapeutic approaches have been tried to limit the immune-mediated CNS damage in MS by targeting the effector functions of activated Th1 cells and macrophages. One is antigen-non-specific immunosuppressive drugs and treatments such as adrenocorticotrophic hormone, corticosteroid, prednisone, methylprednisone, 2-chlorodeoxyadenosine (Cladribine), mitoxantrone, sulphasalazine, methotrexate, total lymphoid irradiation, and β-interferons. Some immunosuppressants have also been tried, e.g., azathioprine, cyclophosphamide and cyclosporin. Limitations of this approach include a risk of infection during non-specific immunosuppression and the toxic side effects of some of the cytotoxic drugs.

Another approach is the use of antigen-specific immunosuppressive drugs and treatments, such as feeding CNS antigens, such as myelin, to tolerize the encephalitogenic T-cells, injecting pathogenic T-cells (T-cell vaccination) or synthetic T-cell receptor peptides to induce immune-mediated elimination of the pathogenic T-cells, injecting tolerogenic peptides that are related to encephalitogenic peptides of CNS antigens like myelin, and giving intravenous immunoglobulin (IVIg). Limitations of this approach include that autoantigenic epitopes are largely undefined in humans, and these epitopes and TCR sequences may differ between MS patients, and within a single MS patient, as the autoimmune reaction spreads to additional epitopes within one protein and to additional proteins.

Also, cytokine-specific therapies have been studied. Examples include neutralizing antibodies against tumor necrosis factor (TNF), soluble TNF-receptors, soluble interleukin-1 antagonists, and others. Limitations of these approaches include solving the problem of delivering the neutralizing agent in sufficient quantity to the CNS tissue site where it is required, and the immunological side effects of long-term cytokine neutralizing activity.

Current therapies for multiple sclerosis include corticosteroid drugs such as methylprednisolone (Solumedrol®) to alleviate the symptoms of acute episodes, muscle relaxants such as tizanidine hydrochloride (Zanaflex®), as well as other biomolecules such as glatiramer acetate (Copaxone®), mitoxantrone (Novantrone®). In particular, β-interferons (IFN-β) have been tested and approved by the U.S. Food and Drug Administration (FDA) as an MS therapy, e.g., interferon-β1a (Avonex®, Rebif®) or interferon-β1b (Betaseron®). Other drugs, e.g., τ-interferon (see, e.g., U.S. Pat. No. 6,060,450), vitamin D analogs, e.g., 1,25(OH)₂D₃ (see, e.g., U.S. Pat. No. 5,716,946), IFN-β-2 (U.S. Pat. Publication No. 20020025304), spirogermaniums, (see, e.g., U.S. Pat. No. 4,654,333), prostaglandins, e.g., latanoprost, brimonidine, PGE1, PGE2 or PGE3. (see, e.g., U.S. Pat. Publication No. 20020004525), tetracyclines and derivatives thereof, e.g., minocycline, doxycycline (U.S. Pat. Publication No. 20020022608), are known.

### SUMMARY OF THE INVENTION

The present invention provides a more effective method of treatment for multiple sclerosis, and pharmaceutical compositions for treating MS which may be used in such methods. A class of agents—statins—repress Class II-mediated T-lymphocyte Activation and consequently act as immunomodulators and anti-inflammatory agents. In an embodiment, the invention relates to methods for treating multiple sclerosis through the administration of one or more statins, and even more advantageously, in combination with other multiple sclerosis agents or treatments, such as interferon-βs or copaxone. The inventors have discovered that statins affect induction of MHC-class II expression by IFN-γ and thus T cell activation. This unexpected effect provides a scientific rationale for the use of statins as novel immunomodulators, in particular as an MS therapy. Moreover, the role of statins in repressing T lymphocyte activation makes them very useful as anti-inflammatory agents.

In one embodiment, methods of treating multiple sclerosis are disclosed, wherein a statin and a second multiple sclerosis drug are administered to a subject having multiple sclerosis, such that the multiple sclerosis is treated or at least partially alleviated. The statin and second multiple sclerosis drug may be administered as part of a pharmaceutical composition, or as part of a combination therapy. In another embodiment, a patient is diagnosed, e.g., to determine if treatment is necessary, whereupon a combination therapy in accordance with the invention is administered to treat the patient. The amount of statin and/or a second multiple sclerosis drug is typically effective to reduce symptoms and to enable an observation of a reduction in symptoms.

Advantageously, the statins which may be used in the invention include Compactin, Atorvastatin, Lovastatin, Mevinolin, Pravastatin, Fluvastatin, Mevastatin, visastatin/Rosuvastatin, Velostatin, Cerivastatin, Simvastatin, Synvinolin, Rivastatin (sodium 7-(4-fluorophenyl)-2,6-diisoprop-yl-5-methoxymethylpyridin-3-yl)-3,5-dihydroxy-6-heptanoate), itavastatin/pitavastatin, pharmaceutically acceptable salts and esters thereof, and combinations thereof. Statins may be administered at a dosage of generally between about 1 and about 500 mg/day, more preferably from about 10 to about 40, 50, 60, 70 or 80 mg/day, advantageously from about 20 to about 40 mg per day. Particularly advantageous statins for use in the invention are those having lipophilic properties, e.g., Compactin, Atorvastatin, Lovastatin, Fluvastatin, Mevastatin, Cerivastatin, or Siravastatin. Atorvastatin is more particularly preferred.

The second multiple sclerosis drug which may be used in the pharmaceutical compositions, methods and combination therapies of the invention include β-interferons, glatiramer acetate, interferon-τ, spirogermaniums (e.g., N-(3-dimethylaminopropyl)-2-aza-8,8-dimethyl-8-germanspiro[4:5]decane, N-(3-dimethylaminopropyl)-2-aza-8,8-diethyl-8-germaspiro[4:5]decane, N-(3-dimethylaminopropyl)-2-aza-8,8-dipropyl-8-germaspiro[4:5]decane and N-(3-dimethylaminopropyl)-2-aza-8,8-dibutyl-8-germaspiro[4:5]decane), vitamin D analogs, prostaglandins (e.g., latanoprost, brimonidine, PGE1, PGE2 and PGE3), tetracyclines (e.g., minocycline and doxycycline), adrenocorticotrophic hormone, corticosteroid, prednisone, methylprednisone, 2-chlorodeoxyadenosine, mitoxantrone, sulphasalazine, methotrexate, azathioprine, cyclophosphamide, cyclosporin, and tizanidine hydrochloride.

Preferably, the second multiple sclerosis drug is an β-interferon, e.g., interferon-β1a, interferon-β1b, or interferon-β2; or glatiramer acetate. If interferon-β1a (i.e., Avonex®) is used, it may be administered at a dosage of about 33 μg (6 MIU), preferably intramuscularly, once a week, or alternately, at a dosage of about 60 μg (12 MIU), preferably intramuscularly, preferably once a week. If interferon-β1a (i.e., Rebif®) is used, it may be administered at a dosage of about 8 μg (2 MIU) to about 50 μg, preferably 22 μg (6 MIU), and most preferably 44 μg (12 MIU), preferably subcutaneously and preferably three times a week. If interferon-β1b (e.g., Betaseron®) is used, it may be administered at a dosage of about 50 μg (1.6 MIU) to about 250 μg (8 MIU), preferably subcutaneously and preferably every other day. If glatiramer acetate (e.g., Copaxone®) is used, it may be administered at a dosage of about 20 mg to about 30 mg, e.g., 20, 25 or 30 mg, preferably 25 mg, preferably administered subcutaneously, preferably daily.

In one embodiment, a combination therapy for MS includes atorvastatin and β-interferon, for treating a patient in need of MS treatment. In another embodiment, a combination therapy for MS includes lovastatin and β-interferon, for treating a patient in need of MS treatment. In an embodiment, a combination therapy for MS includes pravastatin and β-interferon, for treating a patient in need of MS treatment. In another embodiment, a combination therapy for MS includes fluvastatin and β-interferon, for treating a patient in need of MS treatment. In one embodiment, a combination therapy for MS includes mevastatin and β-interferon, for treating a patient in need of MS treatment. In an embodiment, a combination therapy for MS includes rosuvastatin and β-interferon, for treating a patient in need of MS treatment. In yet another embodiment, a combination therapy for MS includes velostatin and β-interferon, for treating a patient in need of MS treatment. In one embodiment, a combination therapy for MS includes cerivastatin and β-interferon, for treating a patient in need of MS treatment. In yet another embodiment, a combination therapy for MS includes itavastatin and β-interferon, for treating a patient in need of MS treatment.

Administration of the therapies and combination therapies of the invention may be (both or individually) orally, topically, subcutaneously, intramuscularly, or intravenously.

The invention further relates to kits for treating patients having multiple sclerosis, comprising a therapeutically effective dose of an agent for treating or at least partially alleviating the symptoms of multiple sclerosis (e.g., β-interferons, glatiramer acetate, interferon-γ, spirogermaniums, vitamin D analogs, prostaglandins, tetracyclines, adrenocorticotrophic hormone, corticosteroid, prednisone, methylprednisone, 2-chlorodeoxyadenosine, mitoxantrone, sulphasalazine, methotrexate, azathioprine, cyclophosphamide, cyclosporin, and tizanidine hydrochloride), and a statin, either in the same or separate packaging, and instructions for its use.

Pharmaceutical compositions comprising a statin and a second multiple sclerosis drug, in an effective amount(s) to treat multiple sclerosis, are also included in the invention.

### BRIEF DESCRIPTION OF THE DRAWING

FIG. 1 is a series of graphs showing that statins decreased IFN-γ induced MHC class II protein expression on human endothelial cells and macrophages. FIGS. 1a to 1f are graphs showing flow cytometric analyses for MHC class II proteins (a-e) and MHC class I (f). FIG. 1a is a flow cytometric analysis achieved on human vascular endothelial cells (ECs) treated with IFN-γ (500 U/ml, 48 hrs) alone (bold line), or with Atorvastatin 10 μM (left dotted line), Lovastatin 10 μM (bold dotted line), or Pravastatin 20 μM (right dotted line). FIG. 1b shows flow cytometric analysis achieved on ECs treated with IFN-γ (500 U/ml, 48 hrs) alone (bold line), or with Atorvastatin 40 nM, 0.2 μM, 2 μM, or 10 μM (from right to left dotted lines, respectively). FIG. 1c shows flow cytometric analysis achieved on ECs treated with IFN-γ alone (500 U/ml, 48 hrs) (bold line), or with Atorvastatin (10 μM) and L-mevalonate (100 μM) (dotted line). FIG. 1d shows flow cytometric analysis achieved on human dendritic cells (DC) under control conditions or treated with Atorvastatin 10 μM (dotted line). FIG. 1e shows flow cytometric analysis achieved on the human cell line Ragi under control conditions or treated with Atorvastatin (10 μM, 48 hrs)(dotted line). FIG. 1f shows flow cytometric analysis achieved on ECs treated with IFN-γ (500 U/ml, 48 hrs) alone (bold line), or with Atorvastatin 10 μM (dotted line). For all panels, solid histograms represent MHC class II (a-e) or MHC class I (ƒ) expression under unstimulated conditions. Each panel is a histogram representing cell numbers (y axis) vs. log fluorescence intensity (x axis) for 30,000 viable cells. Similar results were obtained in independent experiments with ECs and DCs from five different donors. FIG. 1g is a graph showing fluorescence analysis (expressed as relative intensity) for MHC class II expression on human macrophages. (1) is cells under unstimulated conditions, (2), (3), (4) and (5) are cells treated with IFN-γ alone (500 U/ml, 48 hrs), or with Atorvastatin (10 μM), Lovastatin (10 μM) or Pravastatin (20 μM), respectively; (6) is cells treated with IFN-γ (500 U/ml, 48 hrs) and stained with secondary antibody only (negative control). Similar results were obtained in separate experiments using macrophages from three different donors.

FIG. 2 is the association of a blot and its graphic representation showing that the effect of statins on IFN-γ induced MHC class II expression is mediated by the transactivator CIITA. FIG. 2a is a reproduction of an RNAse protection assay (RPA) for MHC class II (DR-α) and FIG. 2b is a reproduction of an RNAse protection assay (RPA) for CIITA. Human vascular endothelial cells unstimulated (1), treated with IFN-γ (500 U/ml, 12 hrs) alone (2), or with Atorvastatin (10 μM) (3), Lovastatin (10 μM) (4), Pravastatin (20 μM (5), or Atorvastatin (10 μM) and L-mevalonate (100 μM) (6). GAPDH was used as a control for RNA loading. Quantification of RPA blots is expressed as the ratio of DR-α/GAPDH and CIITA/GAPDH signal for each sample. Similar results were obtained in independent experiments with ECs from four different donors. * p<0.001, ** p<0.02 compared to IFN-γ treated cells (2),*** p<0.001 compared to IFN-γ/Atorvastatin treated cells (3).

FIG. 3 is a comparison of two different functional consequences of inhibition of MHC class II antigens by statins on T lymphocyte activation. The first consequence is shown by means of the histogram representing [³H]Thymidine incorporation measured in allogenic T lymphocytes exposed (5 days) to human ECs (solid bars) or human Mφ (open bars) or pretreated during 48 hrs with IFN-γ (500 U/mL) alone (1,3), or IFN-γ (500 U/mL) with Atorvastatin (10 μM) (2,4). Similar results were obtained in independent experiments with Mφ or ECs from three different donors. *p<0,02 compared to IFN-γ treated cells. The second consequence is shown by means of the histogram representing IL-2 release measured by ELISA in supernatants of allogenic T lymphocytes exposed (48 hrs) to human ECs (solid bars) or Mφ (open bars) pretreated 48 hrs with IFN-γ (500 U/mL) alone (1,3), or IFN-γ (500 U/mL) with Atorvastatin (10 μM) (2,4). Similar results were obtained in independent experiments with Mφ or ECs from four different donors. **p<0,01 compared to IFN-γ treated cells.

FIG. 4 is a combination of a graph and an electrophoretic gel showing that statins specifically decreased the expression of promoter IV of the transactivator CIITA on a transcriptional level. FIG. 4a is a reproduction of an RNAse protection assay (RPA) for exon 1 of the promoter IV-specific form of CIITA (pIV-CIITA). Human vascular endothelial cells (ECs) unstimulated (1), treated with IFN-γ (500 U/ml, 12 hrs) alone (2), or with Atorvastatin (10 μM) (3), Lovastatin (10 μM) (4), Pravastatin (20 μM ) (5), or Atorvastatin (10 μM) and L-mevalonate (100 μM) (6). GAPDH was used as a control for RNA loading. Quantification of RPA blots is expressed as the ratio of pIV-CIITA/GAPDH signal for each sample. Similar results were obtained in independent experiments with ECs from three different donors. * p<0.001, ** p<0.02 compared to IFN-γ treated cells (2), *** p<0.001 compared to IFN-γ/Atorvastatin treated cells (3). FIG. 4b is a graph representing a densitometric analysis of RPA from actinomycin D (Act D) studies showing the effects of Atorvastatin on pIV-CIITA mRNA levels. ECs were pretreated with IFN-γ (500 U/ml, 12 hrs), and then Act D (10 μg/ml) was added alone or with Atorvastatin (10 μM) and RNA analyzed at different time points. Band intensities of pIV-CIITA/GAPDH mRNA ratio were plotted as a semi-log function of time (hours). Data represent mean±SEM of separate experiments with cells from three different donors. FIG. 4c is a blot representing a Western blot analysis (40 μg protein/lane) of ECs treated with IFN-γ (500 U/ml) in the absence or presence of Lovastatin (10 μM) (Lova). Samples were analyzed for the phosphorylated form of Stat1-α (p Stat1-α) at different periods of time (minutes). Actin was used as a control for protein loading. Blots are representative of different experiments obtained with cells from four different donors.

FIG. 5a and FIG. 5b show the chemical structures of exemplary statins used in the methods, combination therapies and pharmaceutical compositions of the invention.

FIG. 6 is the association of a Western Blot and its graphic representation showing that Statins reduce IFN-γ induced CD40 expression on human atheroma-associated cells. Western blot analysis for CD40 (1-8). Human vascular endothelial cells (ECs) under 1 5 unstimulated conditions (1), treated with IFN-γ (500 U/ml, 24 hrs) alone (2), or with Pravastatin (5 μM, 3), or with Lovastatin (5 μM, 4), or with, Atorvastatin (5 μM, 5), or with Simvastatin (5 μM, 6), or with Sirnvastatin (10 μM) and L-mevalonate (200 μM) (7), Raji under unstimulated condition as positive control (8). Similar results were obtained in independent experiments with ECs from three different donors.

FIG. 7 is a Western Blot showing that Atorvastatin decreases IFN-γ induced CD40 protein expression on human atheroma-associated cells in a dose-dependant manner. Western blot analysis for CD40 (1-6). Human vascular endothelial cells (ECs) under unstimulated conditions (1), treated with IFN-γ (500 U/ml, 24 hrs) alone (2), or with Atorvastatin, 5 μM (3), 2 μM (4), 0.4 μM (5), 0.08 μM (6). Similar results were obtained in independent experiments with ECs from three different donors.

FIG. 8 is a series of graph panels showing the functional effect of Statins on CD40 mediated pathways:

a) MCP-1 release measured by ELISA in supernatants of ECs exposed (24 hrs) with normal media (1), CD40L (5 μg/ml) alone (2), or with Pravastatin (5 μg) (3), or with Lovastatin (5 μg) 9 (4), or with Atorvastatin (5 μM) (5), or with Simvastatin (5 μM) (6), or with Simvastatin (5 μM) and L-Mevalonate (200 μM) (7). Similar results were obtained in independent experiments with ECs from four different donors. * p<0.05 3-6 compared to 2, and 7 compared to 6.

b) IL-6 release measured by ELISA in supernatants of ECs exposed (24 hrs) with normal media (1), CD40L (5 μg/ml) (2), or with Pravastatin (5 μg) (3), or with Lovastatin (5 μM) (4), or with Atorvastatin (5 μM) (5), or with Simvastatin (5 μM) (6). Similar results were obtained in independent experiments with ECs from four different donors.* p<0.05 3-5 compared to 2, and 6 compared to 5.

c) IL-8 release measured by ELISA in supernatants of ECs exposed (24 hrs) with normal media (1), CD40L (51 μg/ml) (2), or with Pravastatin (5 μg) (3), or with Lovastatin (5 μM) (4), or with Atorvastatin (5 μM) (5), or with Simvastatin (5 μM) (6). Similar results were obtained in independent experiments with ECs from four different donors. * p<0.05 3-5 compared to 2, and 6 compared to 5.

FIG. 9 is the association of immunostaining and its graphic representation showing that statins reduce CD40 and CD40L expression on human carotid atheroma. A bank of human carotid atheroma, from patients was analyzed by immunostaining for CD40 and CD40L expression (FIG. 9B), 15 patients being treated with a statin for more than 3 months, 13 patients being not treated with. The statins are simvastatin or atorvastatin, at doses comprised between 20 and 40 mg/day. FIG. 9A shows the graphical representation of CD40 staining area for the two groups; FIG. 9C shows the graphical representation of CD40L staining area for the two groups.

FIG. 10 is the graphical representation of the synergistic effect of a combination therapy in accordance with the invention on human saphenous vein endothelial cells, as shown in detail in Example 3.

### DETAILED DESCRIPTION OF THE INVENTION

The features and other details of the invention will now be more particularly described with reference to the accompanying drawings and pointed out in the claims. It will be understood that particular embodiments described herein are shown by way of illustration and not as limitations of the invention. The principal features of this invention can be employed in various embodiments without departing from the scope of the invention. All parts and percentages are by weight unless otherwise specified.

Definitions

For convenience, certain terms used in the specification, examples, and appended claims are collected here.

“Statins” include molecules capable of acting as inhibitors of HMG-CoA reductase, and pharmaceutically acceptable salts and esters thereof. Members of the statin family include both naturally occurring and synthetic molecules, such as Compactin, Atorvastatin, Lovastatin, Mevinolin, Pravastatin, Fluvastatin, Mevastatin, visastatin/Rosuvastatin, Velostatin, Cerivastatin, Simvastatin, Synvinolin, Rivastatin (sodium 7-(4-fluorophenyl)-2,6-diisoprop-yl-5-methoxymethylpyridin-3-yl)-3,5-dihydroxy-6-heptanoate), itavastatin/pitavastatin, pharmaceutically acceptable salts and esters thereof. These molecules also inhibit IFN-γ-induced CIITA expression in appropriate cells, and/or inhibit HMG-CoA reductase. One accepted way to determine whether a given molecule is a statin or not is to determine sterol synthesis inhibition, especially according to the analyzed tissues and cells, e.g., as in Mastemak, K. et al., “A gene encoding a novel RFX-associated transactivator is mutated in the majority of MHC class II deficiency patients” Nat. Genet. 20, 273-277 (1998). Statins include molecules whose structure differs from that of any member of the statin family by 2 or less substitutions or by modification of chemical bonds. Another way to determine whether a given molecule is a statin or not is to measure the capacity of the molecule to inhibit IFN-γ-induced CIITA expression in appropriate cells, such as in the functional assay described below in the examples. A statin may be hydrophilic, like Pravastatin, or lipophilic like Atorvastatin. Lipophilic statins are believed to better penetrate the tissues.

“IFN-γ responsive cells” include cells having a membrane receptor for IFN-γ, and are capable of transducing a signal after binding of IFN-γ. Some cells can be induced to express MHC class II by IFN-γ. The expression of MHC class II genes is considered a secondary response to IFN-γ since a long lag period is required (24 hours for optimal response in some cases) and requires ongoing protein synthesis, since cycloheximide and/or puromycin (agents that inhibit protein synthesis) abrogate IFN-γ-induced MHC class II expression.

“MHC Class II molecules” include heterodimeric glycoproteins that present antigen to CD4⁺ T cells, leading to T cell activation. Cells which are designated “MHC class II positive” express MHC class II molecules either constitutively or in response to stimulation, e.g., by IFN-γ, and have then MHC class II molecules inserted in their cellular membrane.

“Combination therapy” (or “co-therapy”) includes the administration of a statin and a second multiple sclerosis agent as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). “Combination therapy” may, but generally is not, intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical. “Combination therapy” also can embrace the administration of the therapeutic agents as described above in further combination with other biologically active ingredients and non-drug therapies (e.g., surgery or radiation treatment.) Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

A combination therapy for MS may include atorvastatin and β-interferon. In another embodiment, a combination therapy for MS includes lovastatin and β-interferon. In an embodiment, a combination therapy for MS includes pravastatin and β-interferon. In another embodiment, a combination therapy for MS includes fluvastatin and β-interferon. In one embodiment, a combination therapy for MS includes mevastatin and β-interferon. In an embodiment, a combination therapy for MS includes rosuvastatin and β-interferon. In yet another embodiment, a combination therapy for MS includes velostatin and β-interferon. In one embodiment, a combination therapy for MS includes cerivastatin and β-interferon. In yet another embodiment, a combination therapy for MS includes itavastatin and β-interferon.

“Immunomodulators” include agents whose action on the immune system leads to the immediate or delayed enhancement or reduction of the activity of at least one pathway involved in an immune response, whether this response is naturally occurring or artificially triggered; as part of the innate or adaptive immune system; or both. “MHC Class 11-mediated immunomodulators” include immunomodulators whose key action on the immune system involves MHC class II molecules. Immunomodulation may be considered to be significant if for a given population of allogenic T-lymphocytes, T-cell proliferation is reduced or enhanced by at least 10% after exposure to a statin, compared to the level of T-cell proliferation in the same individual without exposure to the same statin. Whether or not the immunomodulation is significant can be tested using the functional assay described below.

“Immunosuppressors” include agents whose action on the immune system leads to the immediate or delayed reduction of the activity of at least one pathway involved in an immune response, whether this response is naturally occurring or artificially triggered; as part of the innate or adaptive immune system; or both. “MHC Class II-mediated immunosuppressors” include immunosuppressors whose key action on the immune system involves MHC class II molecules. Immunosuppression may be considered to be clinically significant if for a given population of T-lymphocytes, T-cell proliferation is reduced by at least 30%, and preferably at least 50%, after exposure to a statin, compared to the level of T-cell proliferation in the same individual without exposure to the same statin. Whether or not the immunosuppression is clinically significant can be tested using the following assay:

i) A sample of IFN-γ-responsive cells, such as monocytes-macrophages or endothelial cells, is recovered from a first individual and divided into two batches, Batch 1 and Batch 2.

ii) Batch 1 of IFN-γ-responsive cells is pre-treated for approximately 48 hours with IFN-γ (500 U/ml) alone. Batch 2 of IFN-γ-responsive cells is pre treated for approximately 48 hours with IFN-γ (500 U/ml) and a statin (10 μM.).

iii) Allogenic T-lymphocytes (for example, peripheral blood lymphocytes (PBL) are recovered from a different donor, and exposed to pre-treated Batch I and Batch 2 of the IFN-γ-responsive cells (=co-incubation) for the appropriate time indicated below.

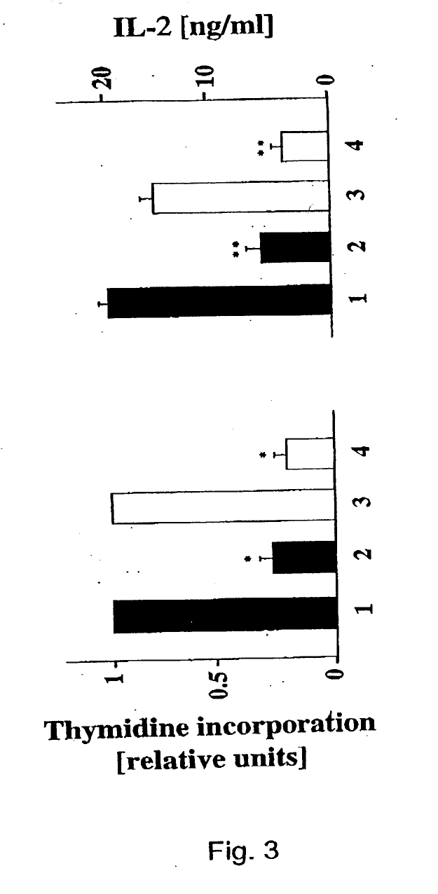

iv) [3^{H}]Thymidine incorporation is measured during the last 24 hours of a 5-day co-incubation period as read-out for T-cell proliferation (see for example FIG. 3).

v) Or Interleukin-2 (IL-2) release is measured after a 2-day co-incubation period as read-out for T-cell proliferation (see for example FIG. 3).

vi) The read-out value for Batch 2 is expressed as a percentage of the read-out for Batch 1. If this value is equal to or less than 70%, preferably equal to or less than 50%, the statin is considered to have a clinically significant immunosuppressive effect.

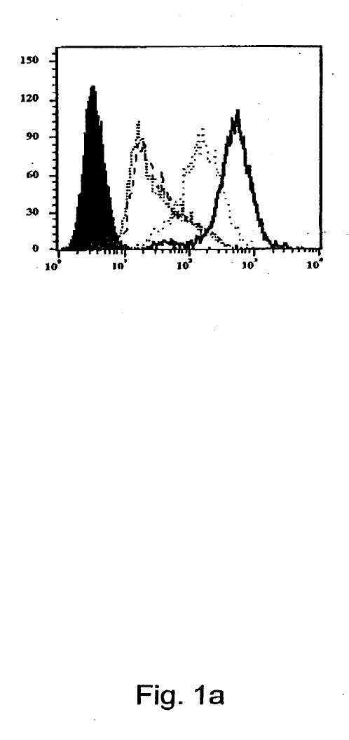

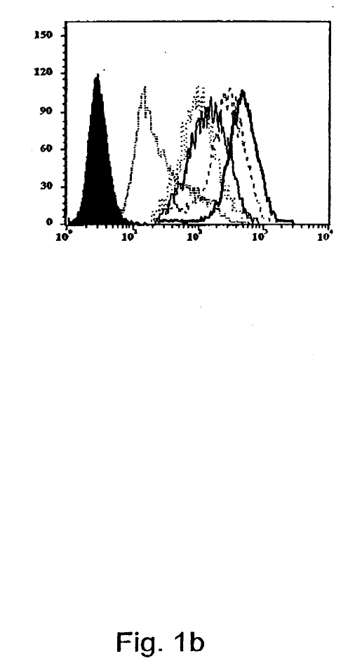

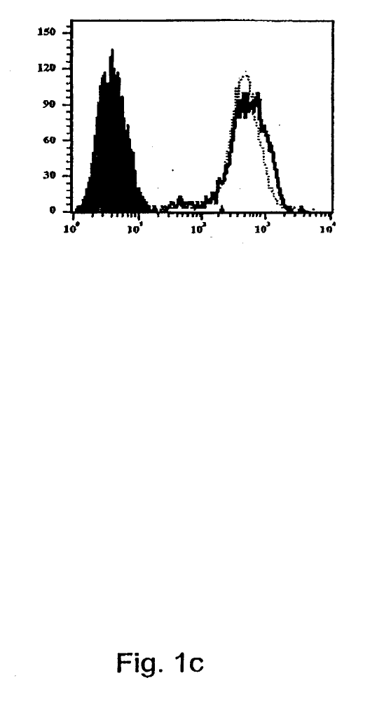

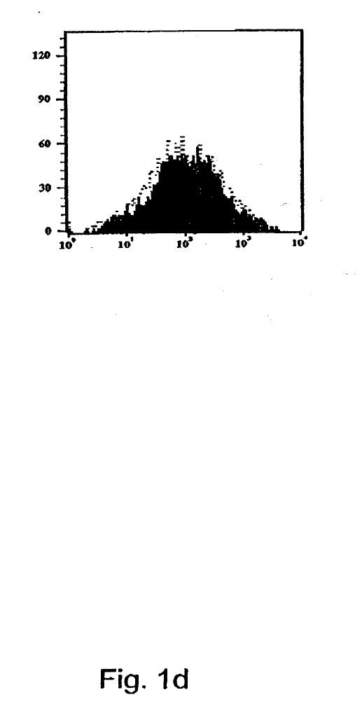

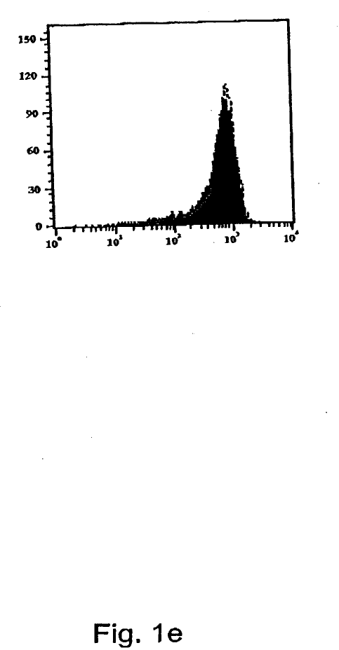

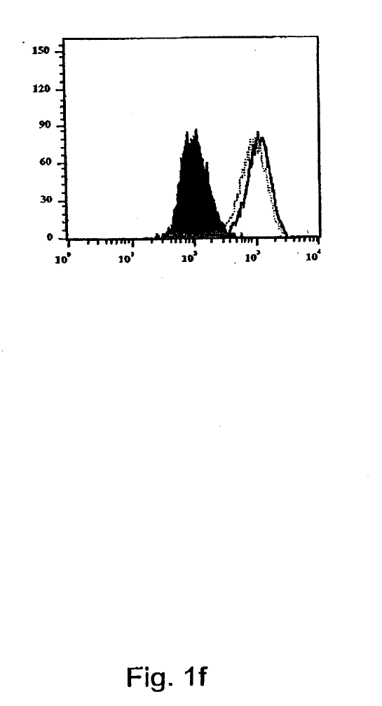

A further means of testing whether the immunosuppressive effect is clinically significant is to carry out the above assessment using Flow Cytometry (see, e.g., FIG. 1).

“Anti-inflammatory agents” are agents capable of reducing or inhibiting inflammation or one of its manifestations, e.g., migration of leucocytes by chemotaxis. “MHC Class II-mediated anti-inflammatory agents” include anti-inflammatory agents whose key action on the immune system involves MHC class II molecules.

“Anti immuno-inflammatory agents” are capable of reducing or inhibiting, partially or totally, immediately or after a delay, inflammation or one of its manifestations as well as other immune responses.

“Detrimental immune response” includes immune responses which are painful or prejudicial to the health of a patient on a long or short-term basis, e.g., immune reactions against self molecules or tissues, or against xenografted tissues or organs.

“Treating”, includes any effect, e.g., lessening, reducing, modulating, or eliminating, that results in the improvement of the condition, disease, disorder, etc.

“Multiple sclerosis symptoms,” includes the commonly observed symptoms of multiple sclerosis, such as those described in Treatment of multiple Sclerosis: Trial Design, Results, and Future Perspectives, ed. Rudick and D. Goodkin, Springer-Verlag, New York, 1992, particularly those symptoms described on pages 48-52.

“Pharmaceutically or pharmacologically acceptable” include molecular entities and compositions that do not produce an adverse, allergic or other untoward reaction when administered to an animal, or a human, as appropriate. “Pharmaceutically acceptable carrier” includes any and all solvents, dispersion media, coatings, antibacterial and antifungal agents, isotonic and absorption delaying agents and the like.

“Immunosuppression” (or “immunomodulation”) becomes clinically desirable in cases where the immune system acts or may act to detrimentally to the health of a patient, and where the shut down or down-regulation of the immune response is then considered by the physician to be useful for the patient's health. Such conditions can be encountered, e.g. in autoimmune diseases including multiple sclerosis, type I diabetes, and rheumatoid arthritis, and, e.g., after an organ transplantation for enhancing tolerance to the graft. Cases in which immunosuppression is clinically required further include the above, and psoriasis and other pathologies. Immunosuppression may also include preventing undesirable immune reactions such as before transplantation.

Autoimmune disorders may be loosely grouped into those primarily restricted to specific organs or tissues and those that affect the entire body. Examples of organ-specific disorders (with the organ affected) include multiple sclerosis (myelin coating on nerve processes), type I diabetes mellitus (pancreas), Hashimotos thyroiditis (thyroid gland), pernicious anemia (stomach), Addison's disease (adrenal glands), myasthenia gravis (acetylcholine receptors at neuromuscular junction), rheumatoid arthritis (joint lining), uveitis (eye), psoriasis (skin), Guillain-Barre Syndrome (nerve cells) and Graves' disease (thyroid). Systemic autoimmune diseases include systemic lupus erythematosus and dermatomyositis.

Major Histocompatibility Complex (MHC) molecules, coded by the HLA gene cluster in man, are involved in many aspects of immunological recognition, including interaction between different lymphoid cells, as well as between lymphocytes and antigen-presenting cells. Major Histocompatibility Complex class II (MHC class II or MHC-II) molecules are directly involved in the activation of T lymphocytes and in the control of the immune response. Although all cells express class I MHC molecules, class II expression is confined to antigen-presenting cells (APCs). These cells are potentially capable of presenting antigen to lymphocytes T-helper which control the development of an immune response. Thus the expression of MHC class II molecules is the key to antigen presentation. Only a limited number of specialized cell types express MHC class II constitutively, numerous other cells become MHC class II positive upon stimulation. The stimulation is usually induction by a cytokine, particularly by interferon gamma (IFN-γ).

Regulation of expression of MHC class II genes is highly complex and this tight control directly affects T lymphocyte activation and thus the control of the immune response. This complex regulation has now been dissected in great detail, thanks to a great extent to a rare human disease of MHC class II regulation, called the Bare Lymphocyte Syndrome (or MHC class II deficiency). Patients with a clinical picture of severe primary immunodeficiency, are affected genetically in one of four distinct transacting regulatory factors, essential for MHC class II gene transcription: whereas RFX5, RFX-AP or RFX-ANK are ubiquitously expressed factors, forming a protein complex that binds to the X box of MHC class II promoters, CITTA (Class II TranActivator) is the general controller of MHC class II expression and its own expression is tightly regulated. Expression of CIITA is controlled by several alternative promoters, operating under distinct physiological conditions. CIITA promoter I controls constitutive expression in dendritic cells, promoter III controls constitutive expression in B and T lymphocytes, while CIITA promoter IV is specifically responsible for the IFN-γ inducible expression of CIITA and thus of MHC class II.

The present invention provides a more effective method of treatment for multiple sclerosis, and pharmaceutical compositions for treating MS which may be used in such methods. A class of agents—statins—repress Class II-mediated T-lymphocyte activation and consequently act as immunomodulators and anti-inflammatory agents. In an embodiment, the invention relates to methods for treating multiple sclerosis through the administration of one or more statins, and even more advantageously, in combination with other multiple sclerosis agents or treatments, such as interferon-βs or copaxone. The inventors have discovered that statins affect induction of MHC-class II expression by IFN-γ and thus T cell activation. This unexpected effect provides a scientific rationale for the use of statins as novel immunomodulators, in particular as an MS therapy. Moreover, the role of statins in repressing T lymphocyte activation makes them very useful as anti-inflammatory agents.

In one embodiment, methods of treating multiple sclerosis are disclosed, wherein a statin and a second multiple sclerosis drug are administered to a subject having multiple sclerosis, such that the multiple sclerosis is treated or at least partially alleviated. The statin and second multiple sclerosis drug may be administered as part of a pharmaceutical composition, or as part of a combination therapy. In another embodiment, a patient is diagnosed, e.g., to determine if treatment is necessary, whereupon a combination therapy in accordance with the invention is administered to treat the patient. The amount of statin and/or a second multiple sclerosis drug is typically effective to reduce symptoms and to enable an observation of a reduction in symptoms.

It has surprisingly been found that in particular, combination therapies of a statin, e.g., Compactin, Atorvastatin, Lovastatin, Mevinolin, Pravastatin, Fluvastatin, Mevastatin, Rosuvastatin, Velostatin, Cerivastatin, Simvastatin, Synvinolin, Rivastatin, itavastatin/pitavastatin, pharmaceutically acceptable salts and esters thereof; and β-interferons are synergistically effective, for example, on T-cell proliferation and thus will be effective in the same manner in treating multiple sclerosis.

Advantageously, the statins which may be used in the invention include Compactin, Atorvastatin, Lovastatin, Mevinolin, Pravastatin, Fluvastatin, Mevastatin, visastatin/Rosuvastatin, Velostatin, Cerivastatin, Simvastatin, Synvinolin, Rivastatin (sodium 7-(4-fluorophenyl)-2,6-diisoprop-yl-5-methoxymethylpyridin-3-yl)-3,5-dihydroxy-6-heptanoate), itavastatin/pitavastatin, pharmaceutically acceptable salts and esters thereof, and combinations thereof. Statins may be administered at a dosage of generally between about 1 and about 500 mg/day, more preferably from about 10 to about 40, 50, 60, 70 or 80 mg/day, advantageously from about 20 to about 40 mg per day. Particularly advantageous statins for use in the invention are those having lipophilic properties, e.g., Compactin, Atorvastatin, Lovastatin, Fluvastatin, Mevastatin, Cerivastatin, or Siravastatin. Atorvastatin is particularly advantageous.

In one embodiment, a combination therapy for MS includes atorvastatin and β-interferon, for treating a patient in need of MS treatment. In another embodiment, a combination therapy for MS includes lovastatin and β-interferon, for treating a patient in need of MS treatment. In an embodiment, a combination therapy for MS includes pravastatin and β-interferon, for treating a patient in need of MS treatment. In another embodiment, a combination therapy for MS includes fluvastatin and β-interferon, for treating a patient in need of MS treatment. In one embodiment, a combination therapy for MS includes mevastatin and β-interferon, for treating a patient in need of MS treatment. In an embodiment, a combination therapy for MS includes rosuvastatin and β-interferon, for treating a patient in need of MS treatment. In yet another embodiment, a combination therapy for MS includes velostatin and β-interferon, for treating a patient in need of MS treatment. In one embodiment, a combination therapy for MS includes cerivastatin and β-interferon, for treating a patient in need of MS treatment. In yet another embodiment, a combination therapy for MS includes itavastatin and β-interferon, for treating a patient in need of MS treatment.

As for every drug, the dosage is an important part of the success of the treatment and the health of the patient. The degree of efficiency as immunomodulator, immunosuppressor or anti-inflammatory agent depends on the particular statin. In every case, in the specified range, the physician has to determine the best dosage for a given patient, according to his sex, age, weight, pathological state and other parameters.

Depending on the chosen statin, or structurally or functionally equivalent derivative, the amount given to the subject must be appropriate, particularly effective to specifically modulate IFN-γ inducible MHC class II expression, so as to obtain the desired immunosuppressive effects.

Administration may be, e.g., intralesional, intraperitoneal, intramuscular or intravenous injection; infusion; or topical, nasal, oral, ocular or otic delivery. A particularly convenient frequency for the administration of statin or derivative is once a day.

The second multiple sclerosis drug which may be used in the pharmaceutical compositions, methods and combination therapies of the invention include β-interferons, glatiramer acetate, interferon-τ, spirogermaniums (e.g., N-(3-dimethylaminopropyl)-2-aza-8,8-dimethyl-8-germanspiro[4:5]decane, N-(3-dimethylaminopropyl)-2-aza-8,8-diethyl-8-germaspiro[4:5]decane, N-(3-dimethylaminopropyl)-2-aza-8,8-dipropyl-8-germaspiro[4:5]decane and N-(3-dimethylaminopropyl)-2-aza-8,8-dibutyl-8-germaspiro[4:5]decane), vitamin D analogs (e.g., 1,25(OH)₂D₃, (see, e.g., U.S. Pat. No. 5,716,946), prostaglandins (e.g., latanoprost, brimonidine, PGE1, PGE2 and PGE3, see, e.g., U.S. Pat. Publication No. 20020004525), tetracyclines (e.g., minocycline and doxycycline, see, e.g., U.S. Pat. Publication No. 20020022608), adrenocorticotrophic hormone, corticosteroid, prednisone, methylprednisone, 2-chlorodeoxyadenosine, mitoxantrone, sulphasalazine, methotrexate, azathioprine, cyclophosphamide, cyclosporin, and tizanidine hydrochloride.

Advantageously the second multiple sclerosis drug is an β-interferon, e.g., interferon-β1a, interferon-β1b, or interferon-β2; or glatiramer acetate. If interferon-β1a (i.e., Avonex®) is used, it may be administered at a dosage of about 33 μg (6 MIU), preferably intramuscularly, once a week, or alternately, at a dosage of about 60 μg (12 MIU), preferably intramuscularly, preferably once a week. If interferon-β1a (i.e., Rebif®) is used, it may be administered at a dosage of about 8 μg (2 MIU) to about 50 μg, preferably 22 μg (6 MIU), and most preferably 44 μg (12 MIU), preferably subcutaneously and preferably three times a week. If interferon-β1b (e.g., Betaseron®) is used, it may be administered at a dosage of about 50 μg (1.6 MIU) to about 250 μg (8 MIU), preferably subcutaneously and preferably every other day. If glatiramer acetate (e.g., Copaxone®) is used, it may be administered at a dosage of about 20 mg to about 30 mg, e.g., 20, 25 or 30 mg, preferably 25 mg, preferably administered subcutaneously, preferably daily.

If vitamin D compounds are used in the combination therapies of the invention, an effective dose may be about 0.5 to about 50 μg/day for a 160 lb. patient. If the compound is a 1α-hydroxy compound, an effective dose may be about 0.5 to about 10 μg/day for a 160 pound patient. Preferably, the dose is between about 0.75 to about 10 μg/day, more preferably about 3 to about 10 μg/day. The dose is preferably divided between two and three treatments per day.

If spirogermaniums are used in the combination therapies of the invention, they may be intravenously given in doses of about 5 to about 80 mg/m² of body surface, and even doses of 120 mg/m² of body surface, much smaller doses can be administered. A recommended dose of spirogermanium therapy is 1.5 cc intramuscularly of an aqueous solution of 30 mg/ml (45 mg/dose). Such treatment is given twice weekly for the first six weeks and once weekly thereafter until remission is obtained.

If IFN-τ is used in the combination therapies of the invention, it can be administered at rates from about 5×10⁴ to about 20×10⁶ units/day, to about 500×10⁶ units/day or more. In a preferred embodiment, the dosage is about 10⁶ units/day. High doses are preferred for systemic administration.

If IFN-β2 is used in the combination therapies of the invention, it can be administered in effective amounts for treatment, e.g., 1.6 MIU (million International Units according to the international reference standard) and 8 MIU administered subcutaneously on alternate days. Effective amounts can be determined routinely, e.g., by performing a dose-response experiment in which varying doses are administered to target cells to determine an effective amount in achieving the desired purpose. Amounts can be selected based on various factors, including the milieu to which the IFN-β2 is administered (e.g., a patient with a multiple sclerosis, animal model, tissue culture cells, etc.), the site of the cells to be treated, the age, health, gender, and weight of a patient or animal to be treated, etc.

If mitoxantrone is used in the combination therapies of the invention, it can be administered in effective amounts for treatment at about 12 mg/m², given as a short (approximately 5 to 15 minutes) intravenous infusion every 3 months.

As noted above, combination therapies of a statin are part of the invention. The combination therapies of the invention may be administered in any suitable fashion to obtain the desired treatment of multiple sclerosis in the patient. One way in which this may be achieved is to prescribe a regimen of statin(s) so as to “pre-treat” the patient to obtain the immunomodulatory effects of the statins, then follow that up with the second multiple sclerosis agent as part of a specific treatment regimen, e.g., a standard administration of interferon-β1a, e.g., intramuscularly or subcutaneously, to provide the benefit of the co-action of the therapeutic agents. Combination therapies of the invention include this sequential administration, as well as administration of these therapeutic agents, or at least two of the therapeutic agents, in a substantially simultaneous manner. Substantially simultaneous administration can be accomplished, for example, by administering to the subject a single capsule or injection having a fixed ratio of a statin and, e.g., a β-interferon, or in multiple, single capsules or injections. The components of the combination therapies, as noted above, can be administered by the same route or by different routes. For example, a statin may be administered by orally, while the other multiple sclerosis agents may be administered intramuscularly or subcutaneously; or all therapeutic agents may be administered orally or all therapeutic agents may be administered by intravenous injection. The sequence in which the therapeutic agents are administered is not believed to be critical.

Administration of the therapies and combination therapies of the invention may be adminstered (both or individually) orally, topically, subcutaneously, intramuscularly, or intravenously.

The invention further relates to kits for treating patients having multiple sclerosis, comprising a therapeutically effective dose of an agent for treating or at least partially alleviating the symptoms of multiple sclerosis (e.g., β-interferons, glatiramer acetate, interferon-τ, spirogermaniums, vitamin D analogs, prostaglandins, tetracyclines, adrenocorticotrophic hormone, corticosteroid, prednisone, methylprednisone, 2-chlorodeoxyadenosine, mitoxantrone, sulphasalazine, methotrexate, azathioprine, cyclophosphamide, cyclosporin, and tizanidine hydrochloride), and a statin, either in the same or separate packaging, and instructions for its use.

In one embodiment, a kit includes therapeutic doses of atorvastatin and β-interferon, for treating a patient in need of MS treatment, and instructions for use. In another embodiment, a kit includes therapeutic doses of lovastatin and β-interferon, for treating a patient in need of MS treatment, and instructions for use. In an embodiment, a kit includes therapeutic doses of pravastatin and β-interferon, for treating a patient in need of MS treatment, and instructions for use. In another embodiment, a kit includes therapeutic doses of fluvastatin and β-interferon, for treating a patient in need of MS treatment, and instructions for use. In one embodiment, a kit includes therapeutic doses of mevastatin and β-interferon, for treating a patient in need of MS treatment, and instructions for use. In an embodiment, a kit includes therapeutic doses of rosuvastatin and β-interferon, for treating a patient in need of MS treatment, and instructions for use. In yet another embodiment, a kit includes therapeutic doses of velostatin and β-interferon, for treating a patient in need of MS treatment, and instructions for use. In one embodiment, a kit includes therapeutic doses of cerivastatin and β-interferon, for treating a patient in need of MS treatment, and instructions for use. In yet another embodiment, a kit includes therapeutic doses of itavastatin and β-interferon, for treating a patient in need of MS treatment, and instructions for use.

Pharmaceutical compositions comprising a statin and a second multiple sclerosis drug, in an effective amount(s) to treat multiple sclerosis, are also included in the invention. The methods described herein can be carried out either in vivo or in vitro (or ex vivo).

In one embodiment, a pharmaceutical composition includes therapeutic doses of atorvastatin and β-interferon, for treating a patient in need of MS treatment. In another embodiment, a pharmaceutical composition includes therapeutic doses of lovastatin and β-interferon, for treating a patient in need of MS treatment. In an embodiment, a pharmaceutical composition includes therapeutic doses of pravastatin and β-interferon, for treating a patient in need of MS treatment. In another embodiment, a pharmaceutical composition includes therapeutic doses of fluvastatin and β-interferon, for treating a patient in need of MS treatment. In one embodiment, a pharmaceutical composition includes therapeutic doses of mevastatin and β-interferon, for treating a patient in need of MS treatment. In an embodiment, a pharmaceutical composition includes therapeutic doses of rosuvastatin and β-interferon, for treating a patient in need of MS treatment. In yet another embodiment, a pharmaceutical composition includes therapeutic doses of velostatin and β-interferon, for treating a patient in need of MS treatment. In one embodiment, a pharmaceutical composition includes therapeutic doses of cerivastatin and β-interferon, for treating a patient in need of MS treatment. In yet another embodiment, a pharmaceutical composition includes therapeutic doses of itavastatin and β-interferon, for treating a patient in need of MS treatment.

The present invention is suitable for the reduction of multiple sclerosis symptoms. These multiple sclerosis symptoms include perturbations of pyramidal functions, e.g., development of paraparesis, hemiparesis, monoparesis and quadriparesis and the development of monoplegia, paraplegia, quadriplegia, and hemiplegia. The symptoms of multiple sclerosis also include perturbations in cerebellar functions. These perturbations include the development of ataxia, including truncal and limb ataxia. “Paralytic symptoms of multiple sclerosis” includes these perturbations in pyramidal and cerebellar functions.

The symptoms of multiple sclerosis also include changes in brain stem functions including development of nystagmus and extraocular weakness along with dysarthria. Further symptoms include loss of sensory function including decrease in touch or position sense and loss of sensation in limbs. Perturbations in bowel and bladder function, including hesitancy, urgency, retention of bowel or bladder or incontinence, can also occur. Visual functions such as scotoma development are also affected by multiple sclerosis. Cerebral function degeneration, including a decrease in mentation and the development of dementia, is also a symptom.

Preferably, treatment should continue as long as multiple sclerosis symptoms are suspected or observed.

To evaluate whether a patient is benefiting from the (treatment), one would examine the patient's symptoms in a quantitative way, such as by the EDSS, or decrease in the frequency of relapses, or increase in the time to sustained progression, or improvement in the magnetic resonance imaging (MRI) behavior in frequent, serial MRI studies and compare the patient's status measurement before and after treatment. In a successful treatment, the patient status will have improved (i.e., the EDSS measurement number or frequency of relapses will have decreased, or the time to sustained progression will have increased, or the MRI scans will show less pathology).

The compositions and combination therapies of the invention may be administered in combination with a variety of pharmaceutical excipients, including stabilizing agents, carriers and/or encapsulation formulations as described herein.

Aqueous compositions of the present invention comprise an effective amount of the peptides of the invention, dissolved or dispersed in a pharmaceutically acceptable carrier or aqueous medium.

“Pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For human administration, preparations should meet sterility, pyrogenicity, general safety and purity standards as required by FDA Office of Biologics standards.

The compositions and combination therapies of the invention will then generally be formulated for parenteral administration, e.g., formulated for injection via the intravenous, intramuscular, subcutaneous, intralesional, or even intraperitoneal routes. The preparation of an aqueous composition that contains a composition of the invention or an active component or ingredient will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fuingi.

Solutions of active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

Therapeutic or pharmacological compositions of the present invention will generally comprise an effective amount of the component(s) of the combination therapy, dissolved or dispersed in a pharmaceutically acceptable medium. Pharmaceutically acceptable media or carriers include any and all solvents, dispersion media, coatings, antibacterial and antifungal agents, isotonic and absorption delaying agents and the like. The use of such media and agents for pharmaceutical active substances is well known in the art. Supplementary active ingredients can also be incorporated into the therapeutic compositions of the present invention.

The preparation of pharmaceutical or pharmacological compositions will be known to those of skill in the art in light of the present disclosure. Typically, such compositions may be prepared as injectables, either as liquid solutions or suspensions; solid forms suitable for solution in, or suspension in, liquid prior to injection; as tablets or other solids for oral administration; as time release capsules; or in any other form currently used, including cremes, lotions, mouthwashes, inhalants and the like.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The preparation of more, or highly, concentrated solutions for intramuscular injection is also contemplated. In this regard, the use of DMSO as solvent is preferred as this will result in extremely rapid penetration, delivering high concentrations of the active compound(s) or agent(s) to a small area.

The use of sterile formulations, such as saline-based washes, by surgeons, physicians or health care workers to cleanse a particular area in the operating field may also be particularly useful. Therapeutic formulations in accordance with the present invention may also be reconstituted in the form of mouthwashes, or in conjunction with antifungal reagents. Inhalant forms are also envisioned. The therapeutic formulations of the invention may also be prepared in forms suitable for topical administration, such as in cremes and lotions.

Suitable preservatives for use in such a solution include benzalkonium chloride, benzethonium chloride, chlorobutanol, thimerosal and the like. Suitable buffers include boric acid, sodium and potassium bicarbonate, sodium and potassium borates, sodium and potassium carbonate, sodium acetate, sodium biphosphate and the like, in amounts sufficient to maintain the pH at between about pH 6 and pH 8, and preferably, between about pH 7 and pH 7.5. Suitable tonicity agents are dextran 40, dextran 70, dextrose, glycerin, potassium chloride, propylene glycol, sodium chloride, and the like, such that the sodium chloride equivalent of the ophthalmic solution is in the range 0.9 plus or minus 0.2%. Suitable antioxidants and stabilizers include sodium bisulfite, sodium metabisulfite, sodium thiosulfite, thiourea and the like. Suitable wetting and clarifying agents include polysorbate 80, polysorbate 20, poloxamer 282 and tyloxapol. Suitable viscosity-increasing agents include dextran 40, dextran 70, gelatin, glycerin, hydroxyethylcellulose, hydroxmethylpropylcellulose, lanolin, methylcellulose, petrolatum, polyethylene glycol, polyvinyl alcohol, polyvinylpyrrolidone, carboxymethylcellulose and the like.

Upon formulation, therapeutics will be administered in a manner compatible with the dosage formulation, and in such amount as is pharmacologically effective. The formulations are easily administered in a variety of dosage forms, such as the type of injectable solutions described above, but drug release capsules and the like can also be employed.

In this context, the quantity of active ingredient and volume of composition to be administered depends on the host animal to be treated. Precise amounts of active compound required for administration depend on the judgment of the practitioner and are peculiar to each individual.

A minimal volume of a composition required to disperse the active compounds is typically utilized. Suitable regimes for administration are also variable, but would be typified by initially administering the compound and monitoring the results and then giving further controlled doses at further intervals. For example, for parenteral administration, a suitably buffered, and if necessary, isotonic aqueous solution would be prepared and used for intravenous, intramuscular, subcutaneous or even intraperitoneal administration. One dosage could be dissolved in 1 ml of isotonic NaCl solution and either added to 1000 ml of hypodermolysis fluid or injected at the proposed site of infusion, (see for example, Remington's Pharmaceutical Sciences 15th Edition, pages 1035-1038 and 1570-1580).

In certain embodiments, active compounds may be administered orally. This is contemplated for agents which are generally resistant, or have been rendered resistant, to proteolysis by digestive enzymes. Such compounds are contemplated to include chemically designed or modified agents; dextrorotatory peptides; and peptide and liposomal formulations in time release capsules to avoid peptidase and lipase degradation.

Pharmaceutically acceptable salts include acid addition salts and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze drying techniques which yield a powder of the active ingredient plus any additional desired ingredient from a previously sterile-filtered solution thereof.

The preparation of more, or highly, concentrated solutions for direct injection is also contemplated, where the use of DMSO as solvent is envisioned to result in extremely rapid penetration, delivering high concentrations of the active agents to a small area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure.

In addition to the compounds formulated for parenteral administration, such as intravenous or intramuscular injection, other pharmaceutically acceptable forms include, e.g., tablets or other solids for oral administration; liposomal formulations; time-release capsules; and any other form currently used, including cremes.

Additional formulations suitable for other modes of administration include suppositories. For suppositories, traditional binders and carriers may include, for example, polyalkylene glycols or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders.

In certain defined embodiments, oral pharmaceutical compositions will comprise an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tables, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 75% of the weight of the unit, or preferably between 25-60%. The amount of active compounds in such therapeutically useful compositions is such that a suitable dosage will be obtained.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabensas preservatives, a dye and flavoring, such as cherry or orange flavor.

In a preferred mode of action of statins, or functional or structural derivatives, the regulation of IFN-γ-induced CIITA expression is solely achieved by inhibition of the CIITA inducible promoter IV. By “solely achieved” is meant that the statins have no effect, or substantially no effect, on the constitutive expression of CIITA, namely expression regulated by promoters I and III See, e.g., Muhlethaler-Mottet, A. et al., “Expression of MHC Class II molecules in different cellular and functional compartments is controlled by differential usage of multiple promoters of transactivator CIITA”, Embo J. 16, 2851-2860 (1997).

As mentioned above, it is surprisingly the effect of statins as HMG-CoA reductase inhibitors that mediates repression of MHC class II by inhibition of CIITA. Indeed, providing the cell with L-mevalonate—which is the product of HMG-CoA reductase—abolishes inhibition by statins. Thus the regulation is reversible at least partially, and preferably filly, by addition of L-mevalonate.

Inhibition of IFN-γ induced CIITA expression at least partially by acting on the HMG-CoA reductase is an unexpected effect with significant clinical potential; molecules capable of is effecting this can be identified by screening as described. The tested property is the ability to inhibit IFN-γ induced CIITA expression in at least partially reversible manner by addition of L-mevalonate. The cells used for this test must be responsive to stimulation by IFN-γ, preferred cells for this purpose are endothelial cells. IFN-γ and the potential inhibitor molecule are contacted with the cells; the detection of MHC class II expression is then carried out. In particular, this step can be accomplished by incubating the cells with for example fluorophore-conjugated specific antibody and then testing by flow cytometry. The skilled man will be aware of other classical ways to detect MHC-class II expression, for example by performing mixed lymphocytes reaction (allogenic T lymphocytes incubated with IFN-γ and candidate molecule-pretreated human endothelial cells) and assaying T cell proliferation. If the candidate molecule appears to be an efficient inhibitor, the additional property of reversibility is tested in a further step which comprises the addition of L-mevalonate to the previous cell culture and detection of a total or partial reversal of the inhibitory effect. This means that expression of MHC class II molecules is at least partially restored. Methods to assay this expression are the same as above. This method also provides a test for identifying functional equivalents of statins.

Implementation of this screening method leads to the selection of inhibitors of CIITA expression which can be then used as such. Following the mode of selection, their action on CIITA is at least partially reversible by addition of L-mevalonate. Inhibitors found according to this screening method may be useful as medicaments having immunosuppressive and antiinflammatory effects or for example in fundamental biology to determine how L-mevalonate derivatives interfere in stimulation by interferon γ.

The subject treated by anyone of the three mentioned methods is a mammal, more preferably a human. The following properties or applications of these methods will essentially be described for humans although they may also be applied to non-human mammals, e.g., apes, monkeys, dogs, mice, etc. The invention therefore can also be used in a veterinarian context.

A patient population susceptible of being treated by methods of the present invention includes patients who in addition to suffering from a condition involving inappropriate or detrimental immune response, may also suffer from hypercholesterolaemia, or from problems in the metabolism of lipids, particularly LDL (low-density lipoproteins), involving high levels of certain lipids. A particularly preferred group of subjects likely to be treated by one of the three methods is a subject who does not suffer from hypercholesterolaemia, irrespective of whether he has or not other risk factors for heart disease and stroke. “Hypercholesterolaemia” includes where LDL-cholesterol levels above 220 mg/dL, preferably above 190 mg/mL, after diet. In cases where a patient presents risk factors for heart disease or stroke, the ‘threshold’ level beyond which hypercholesterolaemia is considered to occur can be lower, for example down to 160 mg/dL, even down to 130 mg/dL.

The inhibition by statins of MHC class II expression is specific for IFN-γ-induced conditions. This specificity is very advantageous, since the immune system as a whole is not disturbed by statins. As such, this is of great therapeutic interest, since the patient under treatment will still be able to fight opportunistic infections.

The methods can be part of a more general treatment of the subject or can be accompanied by a different treatment. In this case, the statin or derivative can be administered with or without other immunosuppressive drugs. In cases where other immunosuppressive drugs are administered, the statin and the other immunosuppressive drugs may be administered separately, simultaneously or sequentially. In a particular case, the statin is administered in the absence of any other immunosuppressive agents, in particular the statin is not administered in combination with cyclosporin A or cyclophosphamide.

### EXAMPLE 1

Materials and Methods

Reagents. Human recombinant IFN-γ was obtained from Endogen (Cambridge, Mass.). The three statins used in these studies [Atorvastatin, (Parke Davis); Lovastatin (Merck Sharp and Dohme); and Pravastatin (Bristol-Myers Squibb)] are commercially available and were obtained from commercial sources. Mouse anti-human MHC class II and MHC class I fluorescein isothiocyanate-conjugated (FITC) and unconjugated monoclonal antibodies were purchased from Pharmingen (San Diego, Calif.). Cycloheximide, actinomycin and L-mevalonate were purchased from Sigma (St. Louis, Mo.).

Cell isolation and culture. Human vascular endothelial cells (ECs) were isolated from saphenous veins by collagenase treatment (Worthington Biochemicals, Freehold, N.J.), and cultured in dishes coated with gelatin (Difco, Liverpool, England) as described in Mach, F. et al, “Functional CD40 is expressed on human vascular endothelial cells, smooth muscle cells, and macrophage: Implication for CD40-CD40 ligand signaling in atherosclerosis”, Proc. Natl. Acad. Sci. USA. 94, 1931-1936 (1997). Cells were maintained in medium 199 (M199; BioWhittaker, Wokingham, England) supplemented with 100 U/ml penicillin/streptomycin (BioWhittaker), 5% FCS (Gibco, Basel, Switzerland), 100 μg/ml heparin (Sigma) and 50 μg/ml ECGF (endothelial cell growth factor; Pel-Freez Biological, Rogers, Ak.). Culture media and FCS contained less than 40 pg LPS/ml as determined by chromogenic Limulus amoebocyte-assay analysis (QLC-1000; BioWhittaker). Endothelial cells were >99% CD31 positive as characterized by flow cytometry and were used at passages 2-4 for all experiments.

Monocytes were isolated from freshly prepared human peripheral blood mononuclear cells obtained from leukopacs of healthy donors following Ficoll-Hypaque gradient and subsequent adherence to plastic culture flasks (90 min., 37° C.). Monocytes were cultured in RPMI 1640 medium (BioWhittaker) containing 10% FCS for 10 days. Macrophages derived from monocytes were >98% CD64 positive as determined by flow cytometry.

The human Raji cell line (Epstein-Barr virus (EBV)-positive Burkitt lymphoma cell line) obtained from American Type Culture Collection (Rockville, Md.) and the human dendritic cells obtained as described in Arrighi, J. F. et al., “Long-term culture of human CD34(+) progenitors with FLT3-ligand, thrombopoietin, and stem cell factor induces extensive amplification of a CD34(−)CD14(−) and a CD34(−)CD14(+) dendritic cell precursor”, Blood 93, 2244-2252 (1999) were grown in RPMI-1640 medium containing 10% FCS.

Flow cytometry. Cells were incubated with FITC-conjugated specific antibody (60 min, 4° C.) and analyzed in a Becton Dickinson FACScan flow cytometer. At least 100.000 viable cells were analyzed per condition. Data were analyzed using CELLQUEST software (Becton Dickinson).

Inuunolabeling. Cells grown on coverslips were fixed for 5 min with methanol at—20° C. The eoverslips were rinsed and incubated successively with 0.2% Triton X-100 in PBS for 1 hour, 0.5 M NH₄Cl in PBS for 15 min and PBS supplemented with 2% bovine serum albumin (Sigma) for another 30 min. Cells were then incubated overnight with primary antibody (1:200) in 10% normal goat serum (Sigma)/PBS. After rinsing, the coverslips were incubated with secondary antibodies FITC-conjugated (1:1000) for 4 h. All steps were performed at room temperature and in between incubation steps cells were rinsed with PBS. Cells were counterstained with 0.03% Evans blue/PBS. Coverslips were mounted on slides in Vectashield (Vector Laboratories, Burlingame, Calif.). Cells were examined using a Zeiss Axiophot microscope equipped with appropriate filters. Specificity of the immunolabeling was checked for by replacing the primary antibody with PBS.

RNAse protection assays. Total RNA was prepared with Tri reagent (MRC, Inc., Cincinnati, Ohio) according to the manufacturer's instructions. RNAse protection assays with 15 μg of RNA per reaction were carried out as described in Muhlethaler-Mottet, A. et al., “Activation of MHC Class II transactivator CIITA by interferon gamma requires cooperative interaction between Stat1 and USF-1”, Immunity 8, 157-166 (1998), using human probes for MHC class II (DR-α, CIITA, exon 1 of the promoter IV-specific form of CIITA (pIV-CIITA), and GAPDH as a control for RNA loading. Signal quantitation was determined using a phosphoimager analysis system (Bio-Rad, Hercules, Calif.). Levels of DR-α, CIITA, and pIV-CIITA RNA in any given sample were normalized to the GAPDH signal for that sample.

Western blot analysis. Cells were harvested in ice-cold RIPA solubilization buffer, and total amounts of protein were determined using a bicinchoninic acid quantification assay (Pierce, Rockford, Ill.). Fifty μg of total protein/lane were separated by SDS/PAGE under reducing conditions and blotted to polyvinylidene difluoride membranes (Millipore Corp., Bedford, Mass.) using a semi-dry blotting apparatus (Bio-Rad, Hercules, Calif.). Blots were blocked overnight in 5% defatted dry milk/PBS/0.1% Tween, and then incubated for 1 hour at room temperature with primary antibody (1:200) (mouse monoclonal anti-human p-Stat1α Santa Cruz, San Diego, Calif.), or mouse monoclonal anti-human β-actin (1:5000) (Pharmingen) for control of loading. This was followed by a 1 hour incubation with secondary peroxidase-conjugated antibody (1:10′000), (Jackson Immunoresearch, West Grove, Pa.). All steps were performed at room temperature and in between incubation steps cells were rinsed with PBS/0.1% Tween. Immunoreactivity was detected using the enhanced chemiluminescence detection method according to the manufacturer's instructions. (Amersham, Düibendorf, Switzerland), and subsequent exposure of the membranes to x-ray film.

Cytokine assay. Release of IL-2 from T lymphocytes was measured using ELISA kits, as suggested by the manufacturer (R&D, Abington, UK). Experiments were performed in the presence of polymyxin B (1 μg/mL). Antibody binding was detected by adding p-nitrophenyl phosphate (1.39 mg/mL), and absorbance was measured at 405 nm in a Dynatech plate reader. The amount of IL-2 detected was calculated from a standard curve prepared with human recombinant IL-2. Samples were assayed in triplicate.

Results

As part of an exploration of possible interfaces between immune mechanisms and atherogenesis, and to evaluate possible beneficial effects of statins independently of their well-known effect as lipid lowering agents, the effect of statins on various features of the control of MHC class II expression and of subsequent lymphocyte activation was analyzed.

The effect of several statins was studied on the regulation of both constitutive MHC class II expression in highly specialized antigen presenting cells (APC) and inducible MHC class II expression by interferon gamma (IFN-γ) in a variety of other cell types, including primary cultures of human endothelial cells (ECs) and monocyte-macrophages (Mφ).

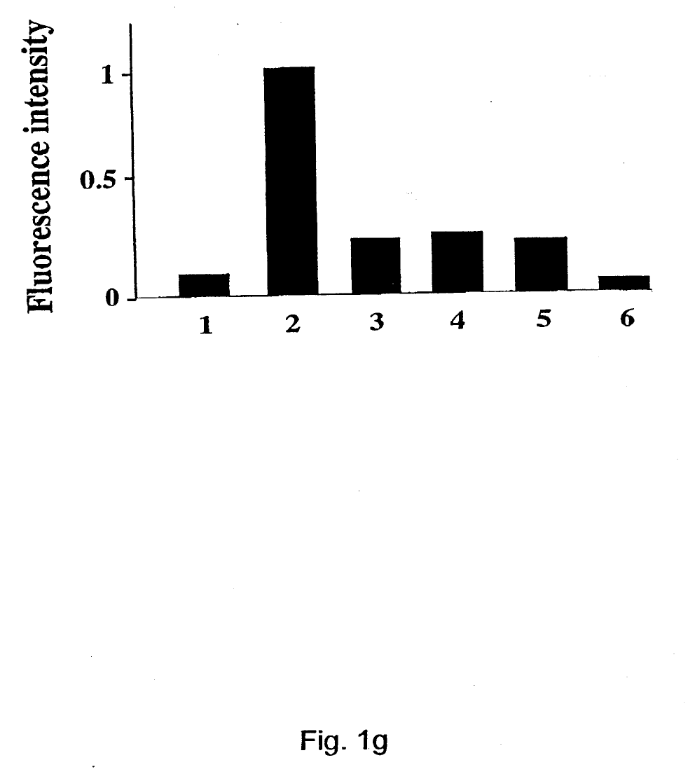

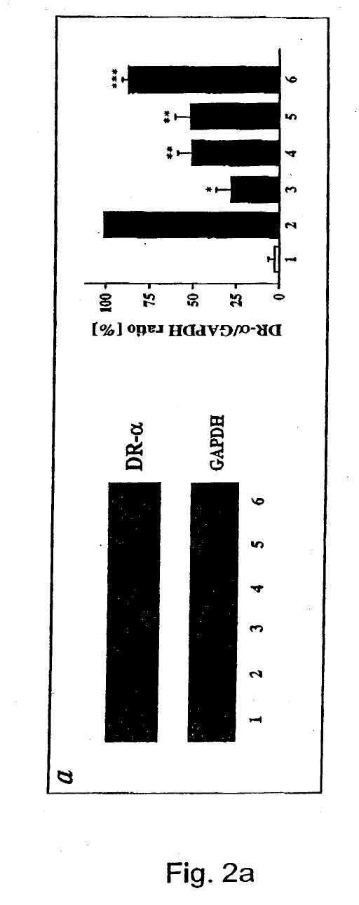

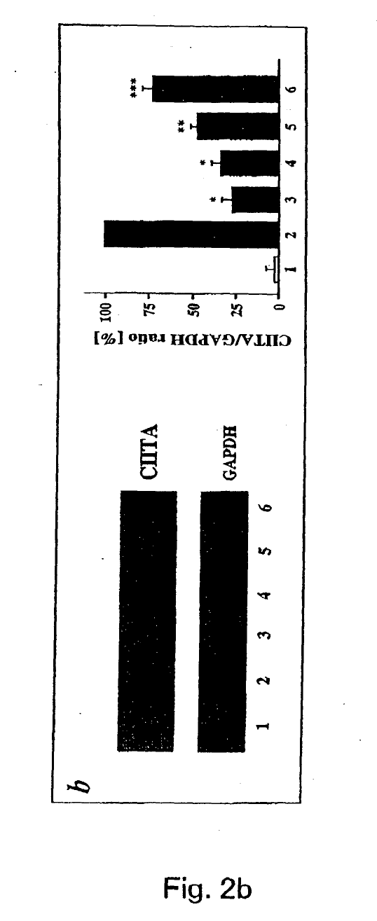

Experiments were performed to monitor cell surface expression (assayed both by FACS, FIGS. 1a-f and by immunofluorescence, FIG. 1g, as well as mRNA levels (RNAse protection assay, FIG. 2a) of MHC class II. These investigations have led to the following conclusions: 1) Statins effectively repress the induction of MHC-II expression by IFN-γ and do so in a dose-dependant manner (FIGS. 1a-b, g). 2) In the presence of L-mevalonate, the effect of statins on MHC class II expression is abolished, indicating that it is indeed the effect of statins as HMG—CoA reductase inhibitors that mediates repression of MHC class II (FIG. 1c). 3) Interestingly, repression of MHC class II expression by statins is highly specific for the inducible form of MHC-II expression and does not concern constitutive expression of MHC-II in highly specialized APCs, such as dendritic cells and B lymphocytes (FIG. 1d,e). 4) This effect of statins is specific for MHC class II and does not concern MHC class I expression (FIG. 1f). 5) In order to investigate functional implications of statin-induced inhibition of MHC class II expression, we performed mix lymphocyte reactions (allogenic T lymphocytes incubated with IFN-γ-pretreated human ECs or Mφ). T cell proliferation could be blocked by anti-MHC class II mAb (monoclonal antibody). Pretreatment of ECs or Mφ with statins represses induction of MHC class II and reduces subsequent T lymphocyte activation and proliferation measured by thymidine incorporation (FIG. 3a) or IL-2 release (FIG. 3b).

The novel effect of statins as MHC class II repressor was also observed and confirmed in other cell types, including primary human smooth muscle cells and fibroblasts, as well as in established cell lines such as ThP1, melanomas and Hela cells. This effect of statins on MHC class II induction is observed with different forms of statins currently used in clinical medicine. Interestingly however, different statins exhibit quite different potency as MHC class II “repressors” (see FIG. 1a). Of the forms tested, the most powerful MHC class II repressor appears to be Atorvastatin. The newly described effect on MHC class II repression can be optimized by screening other members of the statin family, as well as statin analogs.

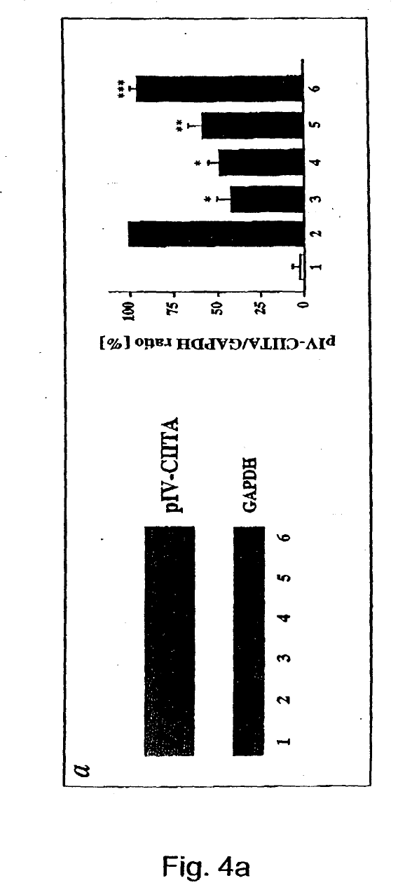

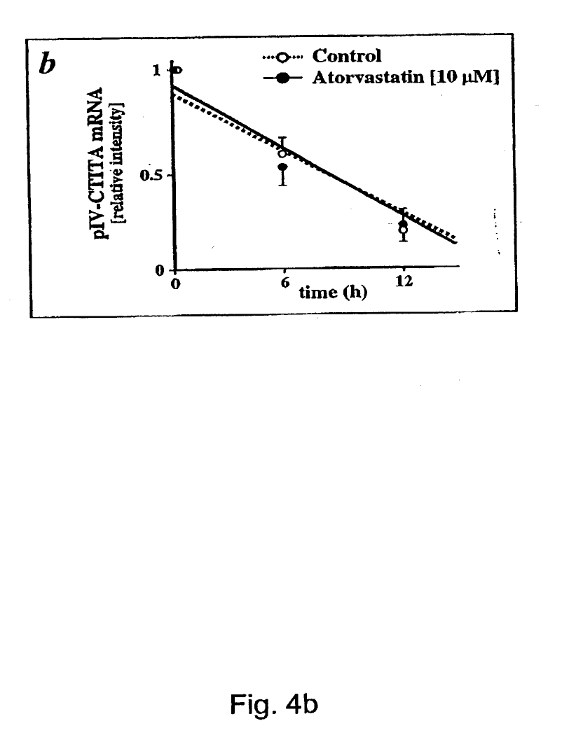

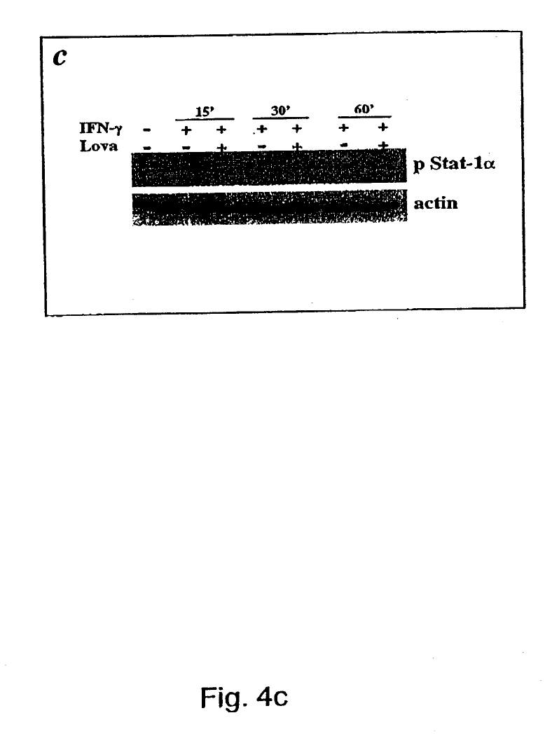

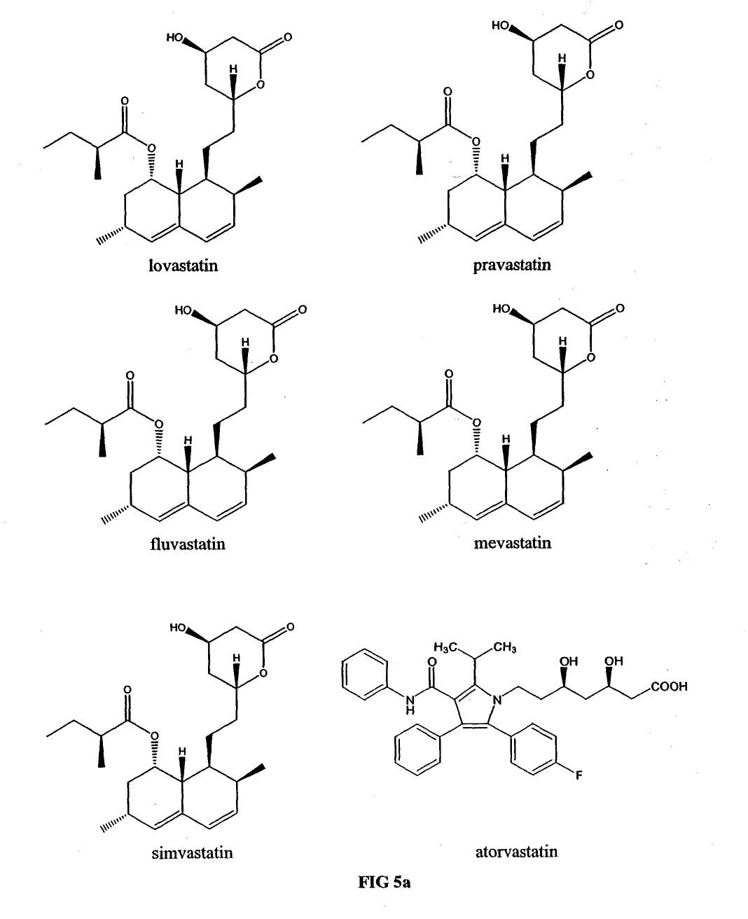

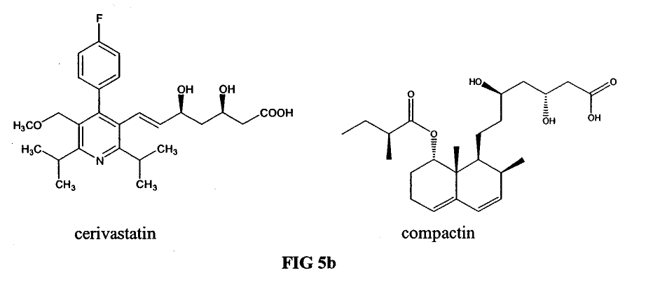

Repression of induction of MHC class II by IFN-γ, in statin treated samples, is paralleled by a reduced induction of CIITA mRNA by IFN-γ (FIG. 2a,b), which points to an inhibition of induction of the CIITA gene by statins. Interestingly, the different degree of repression of CIITA mRNA induction observed with the different forms of statins (FIG. 2b) are reflected in the different levels of repression of MHC class II expression observed with the same drugs (FIG. 1a). This confirms the quantitative nature of the control of CIITA over MHC class II gene activity. Constitutive expression of MHC class II, known to be mediated by CIITA promoters I and III, is not affected by statins (FIGS. 1d,e), suggesting that promoter IV may be their sites of action. Indeed, we also show that induction of expression of the first exon specifically controlled by CIITA promoter IV is affected by statins (FIG. 4a). Finally, the statin effect is transcriptional, as demonstrated by actinomycin D experiments used to block de novo RNA synthesis and explore mRNA half-life (FIG. 4b), and it is direct and does not require de novo protein synthesis, as seen by a lack of effect of cycloheximide experiments.

As expected from the lack of statin effect on MHC class I induction (which is known to require Stat1α), the statin effect reported here is not due to an impairment of Stat1α activation, as phosphorylation and nuclear translocation of Stat1α occurs normally under the effect of statins (FIG. 4c).

### EXAMPLE 2

In this example, statins are shown to reduce CD40 expression.

Materials and Methods

Reagents. Human recombinant IFN-γ was obtained from Endogen (Cambridge). The statins used in these studies Atorvastatin, Simvastatin, Lovastatin, and Pravastatin were obtained from commercial sources. Because endothelial cells lack lactonases to process siravastatin, atorvastatin and lovastatin to their active forms, these agents were chemically activated before their use. Rabbit anti-human CD40 polyclonal Ab, fluorescein isothiocyanate-conjugated (FITC) anti-rabbit Ab, and HRP goat anti-rabbit Ab were purchased from Santa Cruz (Santa Cruz) Jackson ImmunoResearch (West Grovel) and Vector (Burlingame), respectively. FITC-conjugated hamster anti-mouse CD40 monoclonal antibody and FITC-conjugated hamster anti-mouse IgM were purchased by Pharmingen (San Diego). L-mevalonate was purchased from Sigma (St. Louis). Human recombinant CD40 ligand (rCD40L) was a gift from Dr. P. Graber (Serono Pharmaceutical, Geneva, Switzerland) and generated as described by Mazzei. Antibodies for IL-6, IL-8 and MCP-I were obtained from R&D (Oxon).

Cell isolation and culture. Human vascular endothelial cells (ECs) were isolated from saphenous veins and mammary arteries by collagenase treatment (Worthington Biochemicals), and cultured in dishes coated with gelatin (Difco). Cells were maintained in medium 199 (1 4199; BioWhittaker) supplemented with 100 U/ml penicillin/streptomycin (BioWhittaker), 5% FCS (Gibco), 100 μg/ml heparin (Sigma) and 50 μg/ml ECGF (endothelial cell growth factor; Pel-Freez Biological). Human vascular smooth muscle (SMCs) cells were isolated from human saphenous veins and mammary arteries by explant outgrowth, and cultured in DMEM (BioWhittaker) supplemented with 1% L-glutamine (BioWhittaker), 1% penicillin/streptomycin, and 10% FCS. Both cell types were subculture following trypsinization (0.5% trypsin (Worthington Biochemicals)/0.2% EDTA (EM Science)) in P100-culture dishes (Becton Dickinson). Culture media and FCS contained less than 40 pg LPS/ml as determined by chromogenic Limulus amoebocyte-assay analysis (QLC1000; BioWhittaker). ECs and SMCs were >99% CD31 and a-actin (Dako) positive, respectively, as characterized by flow cytometry and were used at passages two to four for all experiments.

The human Raji cell line (Epstein-Barr virus-positive Burkitt lymphoma cell line) obtained from American Type Culture Collection (Rockville) were grown in RPMI-1640 medium containing 10% FCS.

Human monocytes were isolated from freshly prepared human peripheral blood monorruclear cells obtained from leukopacs of healthy donors following Ficoll-Hypaque gradient and subsequent adherence to plastic culture flasks (90 min., 370 C.). Monocytes were cultured in RPMI 1640 medium (BioWhittaker) containing 10% FCS for 10 days. Macrophages (M(D)) derived from monocytes were >98% CD64 positive as determined by flow cytometry.

Mouse monocytes were obtained by peritoneal lavage as described. Animals were on high cholesterol diet (1.25%) for then days before harvesting. Cells were grown in RPMI 1640 medium (BioWhittaker) containing 10% FCS for 10 days.

Western blot analysis. Cells were harvested in ice-cold RIPA solubilization buffer, and total amounts of protein were determined using a bicinchoninic acid quantification assay (Pierce, Rockford, Ill.). Twenty μg of total protein/lane were separated by SDS/PAGE under reducing conditions and blotted to polyvinylidene difluoride membranes (Millipore Corp,, Bedford, Mass.) using a semidry blotting apparatus (Bio-Rad, Hercules, Calif.). Blots were blocked overnight in 5% defatted dry milk/PBS/0.1% Tween, and then incubated for 1 hour at room temperature with primary antibody (1:40) (rabbit polyclonal anti-CD40 Santa Cruz, San Diego, Calif.), or mouse monoclonal anti-human P-actin (1:5000) (Phanningen) for control of loading. This was followed by a 1 hour incubation with secondary peroxidase-conjugated antibody (1:10′000), (Jackson Immunoresearch, West Grove, Pa.). All steps were performed at room temperature and in between incubation steps cells were rinsed with PBS/0.1% Tween. lmmunoreactivity was detected using the enhanced chemiluminescence detection method according to the manufacturer's instructions. (Amersharn, Ddbendorf, Switzerland), and subsequent exposure of the membranes to x-ray film. Analysis of quantification of detection was performed using AIDA software.

Cytokines assay. Release of IL-6, EL-8 and MCP-1 from experiments, was measured using a sandwich-type ELISA as suggested by the manufacturer (R&D system, Abingdon, UK). Experiments were performed in the presence of polymyxin B (1 μg/ml). Antibody binding was detected by adding substrate (R&D), and absorbance measured at 450 nm using a Dynatech plate reader. The amount of IL-6, IL-8 and MCP-I detected was calculated from a standard curve prepared with the recombinant protein. Samples were assayed in duplicates.

Immunolabeling. Human and mice macrophages grown on coverslips, were rinsed and fixed for 15 min with paraformaldehyde (4%) at room temperature (RT). Coverslips were rinsed and cells incubated successively in 0.5M NH₄Cl/PBS for 15 min and PBS supplemented with 2% bovine serum albumin (Sigma) for another 20 min. Human macrophages were then incubated overnight with primary antibody (1:50) in 10% normal goat serum (Sigma)/PBS). Mice macrophages were incubated during 21 hrs with the primary antibody FITC. After rinsing, human macrophages were incubated with secondary antibodies FITC-conjugated (1:800) for 3 hrs. All steps were performed at room temperature and between incubation steps cells were rinsed with PBS. Cells were counterstained with 0.03% Evans blue/PBS. Finally, coverslips were mounted on slides in Vectashield (Vector 32 Laboratories, Burlingame, Calif.). Cells were examined using a Zeiss Axiophot microscope equipped with appropriate filters. Replacement of the primary antibody with PBS/10% normal goat serum or IgM-FITC were used to control the specificity of the immunolabeling of the human macrophages and mice macrophages respectively.

Human immunochemistry. Surgical specimens of human carotid atheroma were obtained by protocols approved by the Investigation Review Committee at the University Hospital Geneva from patients treated or not with the statin Atorvastatin. Serial crystat sections (5 μm) were cut, air dried onto microscope slides (Fisher Scientific), and fixed in acetone at—20′C. for 5 min. Sections were preincubated with blocking buffer (PBS/Tween with 8% of normal horse serum) and then incubated successively with CD40 Ab (goat antihuman)(Santa Cruz) for 1 hour. Finally sections were incubated with biotinylated secondary Ab (45 min; Vector Laboratories) followed by with avidine-biotin-alcaline phosphatase complex (vectastain ABC kit). Antibody binding was visualized with alkaline phosphatase substrate (Vector Laboratories). Cells were not counterstained. Replacing the primary antibody with blocking buffer checked for specificity of the immunolabeling. Analysis of immunochemistery for CD40 was performed with a computer-based quantitative color image analysis system. A color threshold mask for immunostaining was defined to detect the red color by sampling, and all the same threshold was applied to all specimens.

Flow cytometry. Cells were incubated with FITC-conjugated specific antibody (60 min, 40° C.) and analyzed in a Becton Dickinson FACScan flow cytometer. At least 20.000 viable cells were analyzed per condition. Data were analyzed using CELLQUEST software (Becton Dickinson).

Results

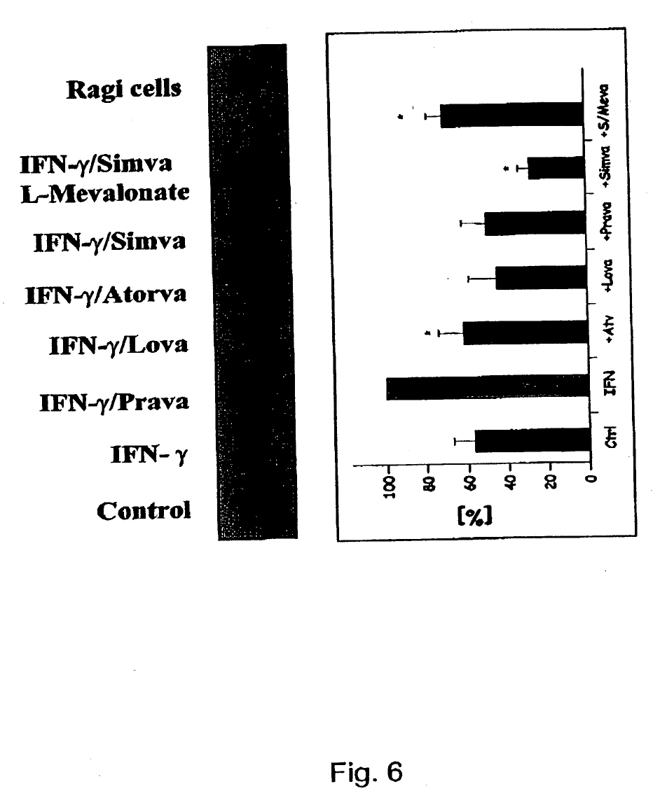

In order to study the effect of statins on IFN-γ induced CD40 expression, confluent vascular endothelial cells (Ecs) were cultured in the presence of 500 U/ml IFN-γ in combination with simvastatin, lovastatin, pravastatin and atorvastatin. Surface CD40 expression was analyzed by Western blotting after 24 hrs. As can be observed in FIG. 6, ECs did express CD40 under resting conditions and IFN-γ treatment induced expression of this molecule. But with co-treatment by IFN-γ and statins, CD40 expression is decreased. Same results were obtained by FACS analysis. Interestingly, statins did not shown any effects by FACS analysis on B lymphocytes (Raji) that constitutively express CD40.

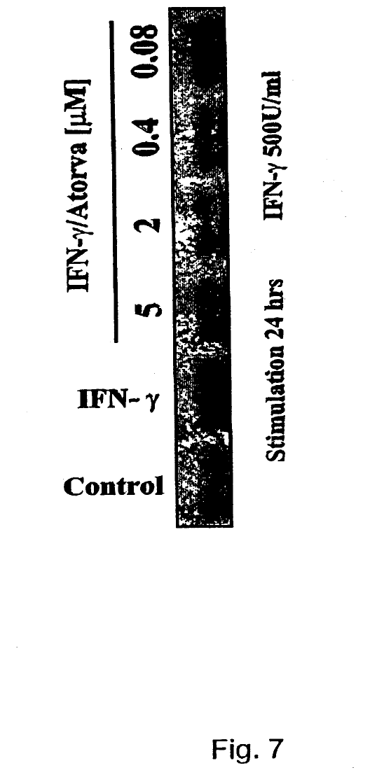

Atorvastatin repressed this induction of CD40 in a dose-dependant manner (FIG. 7). The effect of Atorvastatin was observed over a range of 0.08-0.5 μM. Treatment with Atorvastatin alone had an effect on CD40 expression. HMG-CoA reductase inhibitors, such as Atorvastatin, block the rate-limiting enzyme in the cholesterol synthesis pathway, preventing the production of L-mevalonate. In the presence of L-mevalonate, the effect of Atorvastatin on IFN-γ induced CD40 was markedly reduced.

To investigate the functional consequences of inhibition of CD40 expression by statins on Endothelial Cells activation by CD40L, secreted cytokines were analyzed such as Interleukin-6 (IL-6), interleukin-8 (IL-8), macrophages chemoattractant protein-1 (MCP-1). Addition of an anti-CD4OLmAb blocked the induction of all three secreted cytokines in response to CD40 ligation.

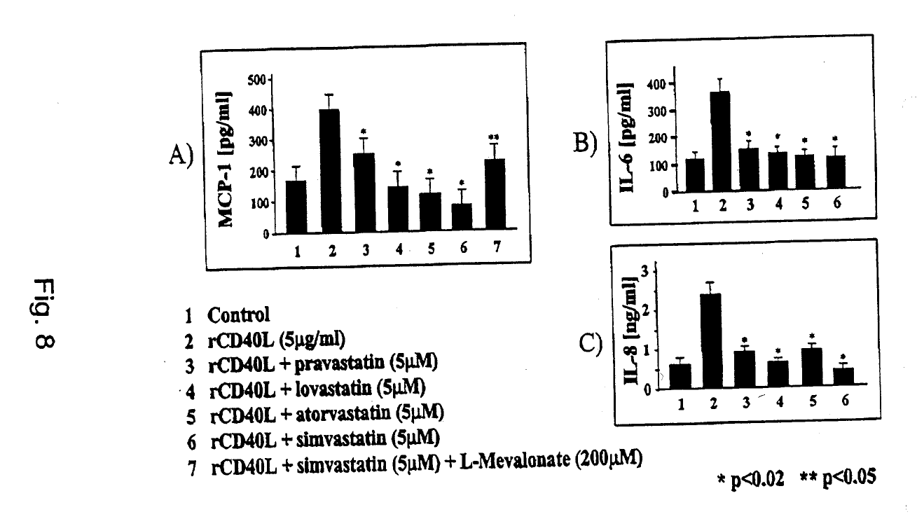

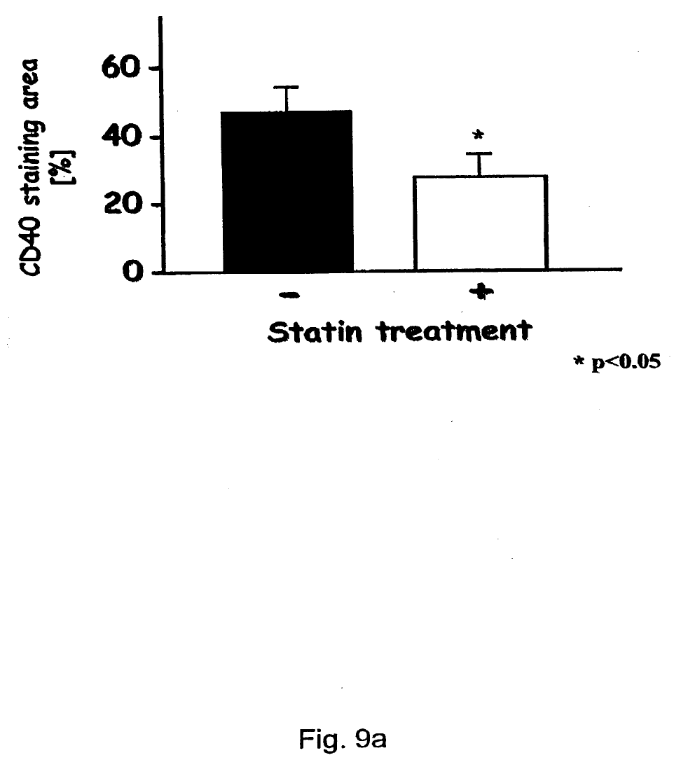

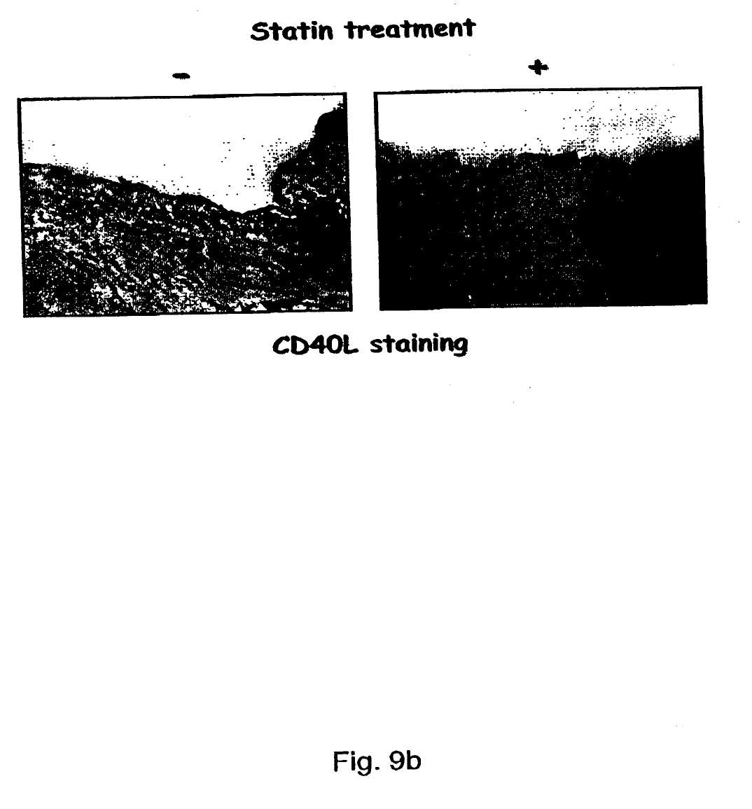

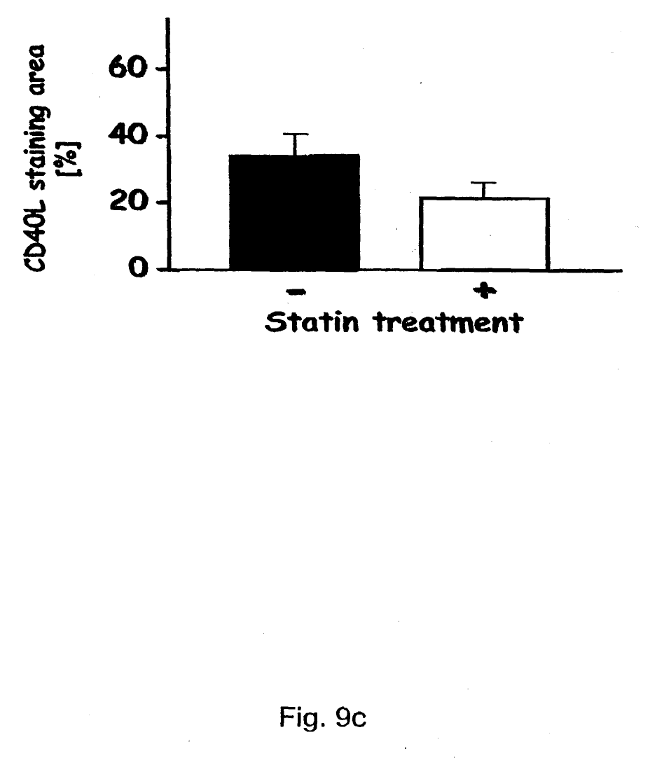

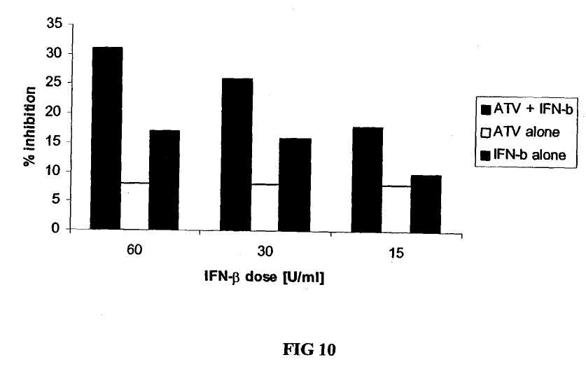

Cytokines were measured by ELISA after 24 hrs. As can be observed in FIGS. 8a, b, c, cytokines are secreted under resting conditions, addition of simvastatin largely reduces the secretion. CD154 treatment induced expression of this molecule. But by CD154 stimulation with statins, CD40 expression is significantly decreased. Addition of L-mevalonate significantly reverses the process.

To determine whereas statins did affect macrophages, an immunofluorescence was performed. The control condition showed a basic level of CD40 which was induced by stimulation with IFN-γ. As expected addition of statins reduced the expression induced by IFN-γ and addition of L-mevalonate.

Arteries carotids plaques were analyzed by immunostaining. Patients under statins treatment present less inflammatory plaques and present less CD40 expression.

Discussion

Increasing evidence supports the central role of CD40L-CD40 signaling pathway responses in several immuno-inflammatory processes, including atherosclerosis, graft-versus-host disease, multiple sclerosis, as well as autoimmune diseases like lupus nephritis, spontaneous autoimmune diabetes, collagen-induced arthritis.

Reducing IFN-γ induced CD40 expression with statins decreases release of chemokines (MCP-1), cytokines (IL-6, EL-8). This might also decrease proagulant activity (tissue factor) (that leads to the thrombus formation), MMPs (that are able to digest the compounds of the matrix and thus participate at the fibrous cap weakening), adhesion molecules as well as B cell activation that could explain plaque stabilization.

In this present invention it is shown that statins decreased IFN-γ induced CD40 expression on vascular cells and thus reduce inflammation induced by the ligation with its ligand.

### EXAMPLE 3

In this example, the synergistic effect of a combination therapy of a statin and IFN-β, on MHC Class II expression is demonstrated.

Effect of atorvastatin (ATV) and interferon-β (IFN-β) combination treatment on inhibition of MHC Class H expression. Human saphenous vein endothelial cells (HSVEC) were cultured and induced with 500 U/ml interferon-γ (R&D Systems) in the presence of atorvastatin alone (40 nM), interferon-β alone (R&D Systems), or in combination as indicated. Forty eight hours later, HSVECs were collected and analyzed for cell surface expression of human MHC class II by FACS. Maximal and minimal MHC class II expression was determined after induction with or without interferon-g alone, respectively. Results are expressed as % inhibition of MHC class II expression. A representative experiment is shown (n=2) in FIG. 10.

As seen in FIG. 10, at doses of 15 and 30 U/ml of interferon-β, the percentage inhibition of MHC class II expression is greater for the combination of atorvastatin and interferon, and the higher dose of 60 U/ml, the synergistic effect of the combination is more clear.

### EXAMPLE 4

In this example, a comparative study of treatment regimens for MS is described.

Treatment with Atorvastatin and/or Avonex:

Patients and Methods

Patients. Patients eligible for this study would include IFN-naïve patients, between the ages of 18-55, diagnosed within the past 2 years with Relapsing-remitting MS (RR-MS). Patients will have evidence of demylination on MRI scanning of the brain and will have an Extended Disability Status Scale (EDSS) score between 0 and 3.5.

Treatment. Patients will be randomized to receive 1 of 4 study arms: Arm 1: Atorvastatin 50 mg/kg oral once per day. Arm 2: Avonex 30 μg IM once weekly. Arm 3: Atorvastatin plus Avonex. Arm 4: Placebo. The study will last a total of 24 weeks.

Study design. Treatment, Double-Blind, Efficacy Study.

Study assessments. The initial screening assessment includes a complete neurologic and medical history, physical and neurologic examination, including the extended disability status scale (EDSS), Ambulation Index (AI), disease steps (DS) scale MS functional composite score, PASAT, 9 hole peg test, and the 25 foot walking time. A 12-lead electrocardiogram (EKG) and chest x-ray will be performed. Serum chemistry is assessed as well as electrolyte and thyroid stimulating hormone (TSH) levels. A brain MRI (with and without gadolinium), urinalysis, and urine pregnancy test (for women of reproductive potential) is performed. Blood is collected for mechanistic studies. Neurologic examination and MRI scans are repeated on study day 1. Patients return to the study center for scheduled follow-up every 4 weeks during the initial 24-week treatment period and also at 36 and 48 weeks. Detailed neurologic assessments by the evaluating physician, including FS and EDSS scoring, are performed at baseline, 12, 24, 36, and 48 weeks, and as needed for relapse assessment. Blood samples are obtained serially for hematologic, biochemical, and thyroid function testing and for determination of neutralizing antibody (Nab) titers. A relapse is defined as the appearance of a new symptom or worsening of an old symptom, accompanied by an appropriate objective finding on neurologic examination by the blinded evaluator, lasting at least 24 hours in the absence of fever and preceded by at least 30 days of clinical stability or improvement. MRI scans are done on study day 1, and every 4 weeks up to week 24. At week 48, a final scan is performed qualifying scans before study initiation. The primary endpoint is the proportion of patients remaining free of relapses during the 24 weeks.

Results

Of the patients chosen for the study, 25 are randomized to receive Atorvastatin, 25 are randomized to receive Avonex, 25 are randomized to receive a combination therapy and 25 are randomized to receive a placebo. Patients taking Atorvastatin with Avonex will exhibit a decrease in the number of relapses and MRI abnormalities compared with patients treated with either Atorvastatin or Avonex alone.

Treatment with Atorvastatin and/or Copaxone:

Patients and Methods

Patients. Patients eligible for this study would include IFN-naïve patients, between the ages of 18-55, diagnosed within the past 2 years with Relapsing-remitting MS (RR-MS). Patients will have evidence of demylination on MRI scanning of the brain and will have an Extended Disability Status Scale (EDSS) score between 0 and 3.5.

Treatment. Patients will be randomized to receive 1 of 4 study arms: Arm 1: Atorvastatin 50 mg/kg oral once per day. Arm 2: Copaxone 20 mg SC once per day. Arm 3: Atorvastatin plus Copaxone. Arm 4: Placebo. The study will last a total of 24 weeks.

Study design. Treatment, Double-Blind, Efficacy Study.

Study assessments. The initial screening assessment includes a complete neurologic and medical history, physical and neurologic examination, including the extended disability status scale (EDSS), Ambulation Index (AI), disease steps (DS) scale MS functional composite score, PASAT, 9 hole peg test, and the 25 foot walking time. A 12-lead electrocardiogram (EKG) and chest x-ray will be performed. Serum chemistry is assessed as well as electrolyte and thyroid stimulating hormone (TSH) levels. A brain MRI (with and without gadolinium), urinalysis, and urine pregnancy test (for women of reproductive potential) is performed. Blood is collected for mechanistic studies. Neurologic examination and MRI scans are repeated on study day 1. Patients return to the study center for scheduled follow-up every 4 weeks during the initial 24-week treatment period and also at 36 and 48 weeks. Detailed neurologic assessments by the evaluating physician, including FS and EDSS scoring, are performed at baseline, 12, 24, 36, and 48 weeks, and as needed for relapse assessment. Blood samples are obtained serially for hematologic, biochemical, and thyroid function testing and for determination of neutralizing antibody (Nab) titers. A relapse is defined as the appearance of a new symptom or worsening of an old symptom, accompanied by an appropriate objective finding on neurologic examination by the blinded evaluator, lasting at least 24 hours in the absence of fever and preceded by at least 30 days of clinical stability or improvement. MRI scans are done on study day 1, and every 4 weeks up to week 24. At week 48, a final scan is performed qualifying scans before study initiation. The primary endpoint is the proportion of patients remaining free of relapses during the 24 weeks.

Results

Of the patients chosen for the study, 25 are randomized to receive Atorvastatin, 25 are randomized to receive Copaxone, 25 are randomized to receive a combination therapy and 25 are randomized to receive a placebo. Patients taking Atorvastatin with Copaxone will exhibit a decrease in the number of relapses and MRI abnormalities compared with patients treated with either Atorvastatin or Copaxone alone.

Treatment with Atorvastatin and/or Rebif:

Patients and Methods

Patients. Patients eligible for this study would include IFN-naïve patients, between the ages of 18-55, diagnosed within the past 2 years with Relapsing-remitting MS (RR-MS). Patients will have evidence of demylination on MRI scanning of the brain and will have an Extended Disability Status Scale (EDSS) score between 0 and 3.5.

Treatment. Patients will be randomized to receive 1 of 4 study arms: Arm 1: Atorvastatin 50 mg/kg oral once per day. Arm 2: Rebif 22 μg SC three times per week. Arm 3: Atorvastatin plus Rebif. Arm 4: Placebo. The study will last a total of 24 weeks.

Study design. Treatment, Double-Blind, Efficacy Study.

Study assessments. The initial screening assessment includes a complete neurologic and medical history, physical and neurologic examination, including the extended disability status scale (EDSS), Ambulation Index (AI), disease steps (DS) scale MS functional composite score, PASAT, 9 hole peg test, and the 25 foot walking time. A 12-lead electrocardiogram (EKG) and chest x-ray will be performed. Serum chemistry is assessed as well as electrolyte and thyroid stimulating hormone (TSH) levels. A brain MRI (with and without gadolinium), urinalysis, and urine pregnancy test (for women of reproductive potential) is performed. Blood is collected for mechanistic studies. Neurologic examination and MRI scans are repeated on study day 1. Patients return to the study center for scheduled follow-up every 4 weeks during the initial 24-week treatment period and also at 36 and 48 weeks. Detailed neurologic assessments by the evaluating physician, including FS and EDSS scoring, are performed at baseline, 12, 24, 36, and 48 weeks, and as needed for relapse assessment. Blood samples are obtained serially for hematologic, biochemical, and thyroid function testing and for determination of neutralizing antibody (Nab) titers. A relapse is defined as the appearance of a new symptom or worsening of an old symptom, accompanied by an appropriate objective finding on neurologic examination by the blinded evaluator, lasting at least 24 hours in the absence of fever and preceded by at least 30 days of clinical stability or improvement. MRI scans are done on study day 1, and every 4 weeks up to week 24. At week 48, a final scan is performed qualifying scans before study initiation. The primary endpoint is the proportion of patients remaining free of relapses during the 24 weeks.

Results

Of the patients chosen for the study, 25 are randomized to receive Atorvastatin, 25 are randomized to receive Rebif, 25 are randomized to receive a combination therapy and 25 are randomized to receive a placebo. Patients taking Atorvastatin with Rebif will exhibit a decrease in the number of relapses and MRI abnormalities compared with patients treated with either Atorvastatin or Rebif alone.

Treatment with Atorvastatin and/or Betaseron:

Patients and Methods

Patients. Patients eligible for this study would include IFN-naïve patients, between the ages of 18-55, diagnosed within the past 2 years with Relapsing-remitting MS (RR-MS). Patients will have evidence of demylination on MRI scanning of the brain and will have an Extended Disability Status Scale (EDSS) score between 0 and 3.5.

Treatment. Patients will be randomized to receive 1 of 4 study arms: Arm 1: Atorvastatin 50 mg/kg oral once per day. Arm 2: Betaseron 0.25 mg SC every other day. Arm 3: Atorvastatin plus Betaseron. Arm 4: Placebo. The study will last a total of 24 weeks.

Study design. Treatment, Double-Blind, Efficacy Study.

Study assessments. The initial screening assessment includes a complete neurologic and medical history, physical and neurologic examination, including the extended disability status scale (EDSS), Ambulation Index (AI), disease steps (DS) scale MS functional composite score, PASAT, 9 hole peg test, and the 25 foot walking time. A 12-lead electrocardiogram (EKG) and chest x-ray will be performed. Serum chemistry is assessed as well as electrolyte and thyroid stimulating hormone (TSH) levels. A brain MRI (with and without gadolinium), urinalysis, and urine pregnancy test (for women of reproductive potential) is performed. Blood is collected for mechanistic studies. Neurologic examination and MRI scans are repeated on study day 1. Patients return to the study center for scheduled follow-up every 4 weeks during the initial 24-week treatment period and also at 36 and 48 weeks. Detailed neurologic assessments by the evaluating physician, including FS and EDSS scoring, are performed at baseline, 12, 24, 36, and 48 weeks, and as needed for relapse assessment. Blood samples are obtained serially for hematologic, biochemical, and thyroid function testing and for determination of neutralizing antibody (Nab) titers. A relapse is defined as the appearance of a new symptom or worsening of an old symptom, accompanied by an appropriate objective finding on neurologic examination by the blinded evaluator, lasting at least 24 hours in the absence of fever and preceded by at least 30 days of clinical stability or improvement. MRI scans are done on study day 1, and every 4 weeks up to week 24. At week 48, a final scan is performed qualifying scans before study initiation. The primary endpoint is the proportion of patients remaining free of relapses during the 24 weeks.

Results

Of the patients chosen for the study, 25 are randomized to receive Atorvastatin, 25 are randomized to receive Betaseron, 25 are randomized to receive a combination therapy and 25 are randomized to receive a placebo. Patients taking Atorvastatin with Betaseron will exhibit a decrease in the number of relapses and MRI abnormalities compared with patients treated with either Atorvastatin or Betaseron alone.

### EXAMPLE 5

In this example, a comparative study of combination therapies for MS is described.

Combination Treatments with Avonex:

Patients and Methods

Patients. Patients are IFN-naïve patients, between the ages of 18-55, diagnosed within the past 2 years with Relapsing-remitting MS (RR-MS). Patients have evidence of demyclination on MRI scanning of the brain and will have an Extended Disability Status Scale (EDSS) score between 0 and 3.5.

Treatment. Patients are randomized to receive one of the following study arms: Arm 1: Atorvastatin 50 mg/kg oral once per day and Avonex 30 μg IM once weekly; Arm 2: Lovastatin 50 mg/kg oral once per day and Avonex 30 μg IM once weekly; Arm 3: Pravastatin 50 mg/kg oral once per day and Avonex 30 μg IM once weekly; Arm 4: Fluvastatin 50 mg/kg oral once per day and Avonex 30 μg IM once weekly; Ann 5: Mevastatin 50 mg/kg oral once per day and Avonex 30 μg IM once weekly; Arm 6: Rosuvastatin 50 mg/kg oral once per day and Avonex 30 μg IM once weekly; Arm 7: Velostatin 50 mg/kg oral once per day and Avonex 30 μg IM once weekly; Arm 8: Cerivastatin 50 mg/kg oral once per day and Avonex 30 μg IM once weekly; Arm 9: Itavastatin 50 mg/kg oral once per day and Avonex 30 μg IM once weekly; Arm 10: Placebo. The study will last a total of 24 weeks.

Study design. Treatment, Double-Blind, Efficacy Study.

Study assessments. The initial screening assessment includes a complete neurologic and medical history, physical and neurologic examination, including the extended disability status scale (EDSS), Ambulation Index (AI), disease steps (DS) scale MS functional composite score, PASAT, 9 hole peg test, and the 25 foot walking time. A 12-lead electrocardiogram (EKG) and chest x-ray will be performed. Serum chemistry is assessed as well as electrolyte and thyroid stimulating hormone (TSH) levels. A brain MRI (with and without gadolinium), urinalysis, and urine pregnancy test (for women of reproductive potential) is performed. Blood is collected for mechanistic studies. Neurologic examination and MRI scans are repeated on study day 1. Patients return to the study center for scheduled follow-up every 4 weeks during the initial 24-week treatment period and also at 36 and 48 weeks. Detailed neurologic assessments by the evaluating physician, including FS and EDSS scoring, are performed at baseline, 12, 24, 36, and 48 weeks, and as needed for relapse assessment. Blood samples are obtained serially for hematologic, biochemical, and thyroid function testing and for determination of neutralizing antibody (Nab) titers. A relapse is defined as the appearance of a new symptom or worsening of an old symptom, accompanied by an appropriate objective finding on neurologic examination by the blinded evaluator, lasting at least 24 hours in the absence of fever and preceded by at least 30 days of clinical stability or improvement. MRI scans are done on study day 1, and every 4 weeks up to week 24. At week 48, a final scan is performed qualifying scans before study initiation. The primary endpoint is the proportion of patients remaining free of relapses during the 24 weeks.

Results

Of the patients chosen for the study, 25 are randomized to receive each of the individual arms of the study. Patients receiving Arms 1 through 9 exhibit a decrease in the number of relapses and MRI abnormalities, compared with patients treated with placebo.

Combination Treatments with Copaxone:

Patients and Methods

Patients. Patients are IFN-naïve patients, between the ages of 18-55, diagnosed within the past 2 years with Relapsing-remitting MS (RR-MS). Patients have evidence of demyelination on MRI scanning of the brain and will have an Extended Disability Status Scale (EDSS) score between 0 and 3.5.

Treatment. Patients are randomized to receive one of the following study arms: Arm 1: Atorvastatin 50 mg/kg oral once per day and Copaxone 20 mg SC once per day; Arm 2: Lovastatin 50 mg/kg oral once per day and Copaxone 20 mg SC once per day; Arm 3: Pravastatin 50 mg/kg oral once per day and Copaxone 20 mg SC once per day; Arm 4: Fluvastatin 50 mg/kg oral once per day and Copaxone 20 mg SC once per day; Arm 5: Mevastatin 50 mg/kg oral once per day and Copaxone 20 mg SC once per day; Arm 6: Rosuvastatin 50 mg/kg oral once per day and Copaxone 20 mg SC once per day; Arm 7: Velostatin 50 mg/kg oral once per day and Copaxone 20 mg SC once per day; Arm 8: Cerivastatin 50 mg/kg oral once per day and Copaxone 20 mg SC once per day; Arm 9: Itavastatin 50 mg/kg oral once per day and Copaxone 20 mg SC once per day; Arm 10: Placebo. The study will last a total of 24 weeks.

Study design. Treatment, Double-Blind, Efficacy Study.

Study assessments. The initial screening assessment includes a complete neurologic and medical history, physical and neurologic examination, including the extended disability status scale (EDSS), Ambulation Index (AI), disease steps (DS) scale MS functional composite score, PASAT, 9 hole peg test, and the 25 foot walking time. A 12-lead electrocardiogram (EKG) and chest x-ray will be performed. Serum chemistry is assessed as well as electrolyte and thyroid stimulating hormone (TSH) levels. A brain MRI (with and without gadolinium), urinalysis, and urine pregnancy test (for women of reproductive potential) is performed. Blood is collected for mechanistic studies. Neurologic examination and MRI scans are repeated on study day 1. Patients return to the study center for scheduled follow-up every 4 weeks during the initial 24-week treatment period and also at 36 and 48 weeks. Detailed neurologic assessments by the evaluating physician, including FS and EDSS scoring, are performed at baseline, 12, 24, 36, and 48 weeks, and as needed for relapse assessment. Blood samples are obtained serially for hematologic, biochemical, and thyroid function testing and for determination of neutralizing antibody (Nab) titers. A relapse is defined as the appearance of a new symptom or worsening of an old symptom, accompanied by an appropriate objective finding on neurologic examination by the blinded evaluator, lasting at least 24 hours in the absence of fever and preceded by at least 30 days of clinical stability or improvement. MRI scans are done on study day 1, and every 4 weeks up to week 24. At week 48, a final scan is performed qualifying scans before study initiation. The primary endpoint is the proportion of patients remaining free of relapses during the 24 weeks.

Results

Of the patients chosen for the study, 25 are randomized to receive each of the individual arms of the study. Patients receiving Arms 1 through 9 exhibit a decrease in the number of relapses and MRI abnormalities, compared with patients treated with placebo.

Combination Treatments with Rebif:

Patients and Methods

Patients. Patients are IFN-naïve patients, between the ages of 18-55, diagnosed within the past 2 years with Relapsing-remitting MS (RR-MS). Patients have evidence of demyelination on MRI scanning of the brain and will have an Extended Disability Status Scale (EDSS) score between 0 and 3.5.

Treatment. Patients are randomized to receive one of the following study arms: Arm 1: Atorvastatin 50 mg/kg oral once per day and Rebif 22 μg SC three times per week; Arm 2: Lovastatin 50 mg/kg oral once per day and Rebif 22 μg SC three times per week; Arm 3: Pravastatin 50 mg/kg oral once per day and Rebif 22 μg SC three times per week; Arm 4: Fluvastatin 50 mg/kg oral once per day and Rebif 22 μg SC three times per week; Arm 5: Mevastatin 50 mg/kg oral once per day and Rebif 22 μg SC three times per week; Arm 6: Rosuvastatin 50 mg/kg oral once per day and Rebif 22 μg SC three times per week; Arm 7: Velostatin 50 mg/kg oral once per day and Rebif 22 μg SC three times per week; Arm 8: Cerivastatin 50 mg/kg oral once per day and Rebif 22 μg SC three times per week; Arm 9: Itavastatin 50 mg/kg oral once per day and Rebif 22 μg SC three times per week; Arm 10: Placebo. The study will last a total of 24 weeks.

Study design. Treatment, Double-Blind, Efficacy Study.

Study assessments. The initial screening assessment includes a complete neurologic and medical history, physical and neurologic examination, including the extended disability status scale (EDSS), Ambulation Index (AI), disease steps (DS) scale MS functional composite score, PASAT, 9 hole peg test, and the 25 foot walking time. A 12-lead electrocardiogram (EKG) and chest x-ray will be performed. Serum chemistry is assessed as well as electrolyte and thyroid stimulating hormone (TSH) levels. A brain MRI (with and without gadolinium), urinalysis, and urine pregnancy test (for women of reproductive potential) is performed. Blood is collected for mechanistic studies. Neurologic examination and MRI scans are repeated on study day 1. Patients return to the study center for scheduled follow-up every 4 weeks during the initial 24-week treatment period and also at 36 and 48 weeks. Detailed neurologic assessments by the evaluating physician, including FS and EDSS scoring, are performed at baseline, 12, 24, 36, and 48 weeks, and as needed for relapse assessment. Blood samples are obtained serially for hematologic, biochemical, and thyroid function testing and for determination of neutralizing antibody (Nab) titers. A relapse is defined as the appearance of a new symptom or worsening of an old symptom, accompanied by an appropriate objective finding on neurologic examination by the blinded evaluator, lasting at least 24 hours in the absence of fever and preceded by at least 30 days of clinical stability or improvement. MRI scans are done on study day 1, and every 4 weeks up to week 24. At week 48, a final scan is performed qualifying scans before study initiation. The primary endpoint is the proportion of patients remaining free of relapses during the 24 weeks.

Results

Of the patients chosen for the study, 25 are randomized to receive each of the individual arms of the study. Patients receiving Arms 1 through 9 exhibit a decrease in the number of relapses and MRI abnormalities, compared with patients treated with placebo.

Combination Treatments with Betaseron:

Patients and Methods

Patients. Patients are IFN-naïve patients, between the ages of 18-55, diagnosed within the past 2 years with Relapsing-remitting MS (RR-MS). Patients have evidence of demyelination on MRI scanning of the brain and will have an Extended Disability Status Scale (EDSS) score between 0 and 3.5.

Treatment. Patients are randomized to receive one of the following study arms: Arm 1: Atorvastatin 50 mg/kg oral once per day and Betaseron 0.25 mg SC every other day; Arm 2: Lovastatin 50 mg/kg oral once per day and Betaseron 0.25 mg SC every other day; Arm 3: Pravastatin 50 mg/kg oral once per day and Betaseron 0.25 mg SC every other day; Arm 4: Fluvastatin 50 mg/kg oral once per day and Betaseron 0.25 mg SC every other day; Arm 5: Mevastatin 50 mg/kg oral once per day and Betaseron 0.25 mg SC every other day; Arm 6: Rosuvastatin 50 mg/kg oral once per day and Betaseron 0.25 mg SC every other day; Arm 7: Velostatin 50 mg/kg oral once per day and Betaseron 0.25 mg SC every other day; Arm 8: Cerivastatin 50 mg/kg oral once per day and Betaseron 0.25 mg SC every other day; Arm 9: Itavastatin 50 mg/kg oral once per day and Betaseron 0.25 mg SC every other day; Arm 10: Placebo. The study will last a total of 24 weeks.

Study design. Treatment, Double-Blind, Efficacy Study.

Study assessments. The initial screening assessment includes a complete neurologic and medical history, physical and neurologic examination, including the extended disability status scale (EDSS), Ambulation Index (AI), disease steps (DS) scale MS functional composite score, PASAT, 9 hole peg test, and the 25 foot walking time. A 12-lead electrocardiogram (EKG) and chest x-ray will be performed. Serum chemistry is assessed as well as electrolyte and thyroid stimulating hormone (TSH) levels. A brain MRI (with and without gadolinium), urinalysis, and urine pregnancy test (for women of reproductive potential) is performed. Blood is collected for mechanistic studies. Neurologic examination and MRI scans are repeated on study day 1. Patients return to the study center for scheduled follow-up every 4 weeks during the initial 24-week treatment period and also at 36 and 48 weeks. Detailed neurologic assessments by the evaluating physician, including FS and EDSS scoring, are performed at baseline, 12, 24, 36, and 48 weeks, and as needed for relapse assessment. Blood samples are obtained serially for hematologic, biochemical, and thyroid function testing and for determination of neutralizing antibody (Nab) titers. A relapse is defined as the appearance of a new symptom or worsening of an old symptom, accompanied by an appropriate objective finding on neurologic examination by the blinded evaluator, lasting at least 24 hours in the absence of fever and preceded by at least 30 days of clinical stability or improvement. MRI scans are done on study day 1, and every 4 weeks up to week 24. At week 48, a final scan is performed qualifying scans before study initiation. The primary endpoint is the proportion of patients remaining free of relapses during the 24 weeks.

Results

Of the patients chosen for the study, 25 are randomized to receive each of the individual arms of the study. Patients receiving Arms 1 through 9 exhibit a decrease in the number of relapses and MRI abnormalities, compared with patients treated with placebo.

### EQUIVALENTS

Those skilled in the art will recognize, or be able to ascertain using no more than routine experimentation, numerous equivalents to the specific procedures described herein. Such equivalents are considered to be within the scope of the present invention and are covered by the following claims. Various substitutions, alterations, and modifications may be made to the invention without departing from the spirit and scope of the invention as defined by the claims. Other aspects, advantages, and modifications are within the scope of the invention. The contents of all references, issued patents, and published patent applications cited throughout this application are hereby filly incorporated by reference. The appropriate components, processes, and methods of those patents, applications and other documents may be selected for the present invention and embodiments thereof.

What is claimed is:

1. A method of treating multiple sclerosis, comprising administering to a subject having multiple sclerosis a combination therapy including a statin and a second multiple sclerosis drug, such that said multiple sclerosis is treated or at least partially alleviated.

2. A method of treating multiple sclerosis, comprising administering to a patient in need thereof a pharmaceutical composition comprising a statin and a second multiple sclerosis drug, in an amount effective to treat said multiple sclerosis in said patient.

3. A method of treating multiple sclerosis, comprising diagnosing a patient in need of treatment and administering to a patient in need thereof a combination therapy including a statin and a second multiple sclerosis drug, such that said multiple sclerosis is treated or at least partially alleviated.

4. The method of claim 1, wherein the amount of said statin and/or said a second multiple sclerosis drug is effective to reduce symptoms and to enable an observation of a reduction in symptoms.

5. A combination therapy for treating multiple sclerosis, comprising administering to a subject having multiple sclerosis a statin and a second multiple sclerosis drug, such that said multiple sclerosis is treated or at least partially alleviated.

6. The method of claim 1, wherein said patient does not suffer from hypercholesterolemia.

7. The method of claim 1, wherein said statin is selected from the group consisting of Compactin, Atorvastatin, Lovastatin, Mevinolin, Pravastatin, Fluvastatin, Mevastatin, visastatin/Rosuvastatin, Velostatin, Cerivastatin, Simvastatin, Synvinolin, Rivastatin (sodium 7-(4-fluorophenyl)-2,6-diisoprop-yl-5-methoxymethylpyridin-3-yl)-3,5-dihydroxy-6-heptanoate), itavastatin/pitavastatin, pharmaceutically acceptable salts and esters thereof, and combinations thereof.

8. The method of claim 1, wherein said second multiple sclerosis drug is selected from the group consisting of β-interferons, glatiramer acetate, interferon-τ, spirogermaniums, vitamin D analogs, prostaglandins, tetracyclines, adrenocorticotrophic hormone, corticosteroid, prednisone, methylprednisone, 2-chlorodeoxyadenosine, mitoxantrone, sulphasalazine, methotrexate, azathioprine, cyclophosphamide, cyclosporin, and tizanidine hydrochloride.

9. The method of claim 8, wherein said second multiple sclerosis drug is an interferon-β or glatiramer acetate.

10. The method of claim 8, wherein said β-interferon is interferon-β1a, interferon-β1b, or interferon-β2.

11. The method of claim 10, wherein said β-interferon is interferon-β1a (AVONEX) administered at a dosage of about 33 μg.

12. The method of claim 11, wherein said interferon-β1a is administered intramuscularly.

13. The method of claim 10, wherein said β-interferon is interferon-β1a (REBIF) administered at a dosage of about 8 to about 50 μg.

14. The method of claim 13, wherein said β-interferon is interferon-β1a administered at a dosage of about 22 μg.

15. The method of claim 13, wherein said β-interferon is interferon-β1a administered at a dosage of about 44 μg.

16. The method of claim 13, wherein said interferon-β1a is administered intramuscularly.

17. The method of claim 10, wherein said β-interferon is interferon-β1b (BETASERON) administered at a dosage of about 25 μg.

18. The method of claim 17, wherein said interferon-β1b is administered subcutaneously.

19. The method of claim 8, wherein said second multiple sclerosis drug is glatiramer acetate (COPAXONE) administered at a dosage of about 20 mg.

20. The method of claim 19, wherein said glatiramer acetate is administered subcutaneously.

21. The method of claim 8, wherein said prostaglandin is selected from the group consisting of latanoprost, brimonidine, PGE1, PGE2 and PGE3.

22. The method of claim 8, wherein said tetracycline is selected from the group consisting of minocycline and doxycycline.

23. The method of claim 8, wherein said spirogermanium is selected from the group consisting of N-(3-dimethylaminopropyl)-2-aza-8,8-dimethyl-8-germanspiro[4:5]decane, N-(3-dimethylaminopropyl)-2-aza-8,8-diethyl-8-germaspiro[4:5]decane, N-(3-dimethylaminopropyl)-2-aza-8,8-dipropyl-8-germaspiro[4:5]decane and N-(3-dimethylaminopropyl)-2-aza-8,8-dibutyl-8-germaspiro[4:5]decane.

24. The method of claim 8, wherein said second multiple sclerosis drug is interferon-τ.

25. The method of claim 1, wherein said treatment is administered orally.

26. The method of claim 1, wherein said treatment is administered topically.

27. The method of claim 1, wherein said treatment is administered subcutaneously.

28. The method of claim 1, wherein said treatment is administered intramuscularly.

29. The method of claim 1, wherein said treatment is administered intravenously.

30. The method of claim 1, wherein the amount of said statin is at least about 10 to 80 mg per day.

31. The method of claim 1, wherein the dose of statin is at least about 10 to 80 mg per day.

32. The method of claim 1, wherein the dose of statin is at least about 10 to 70 mg per day.

33. The method of claim 1, wherein the dose of statin is at least about 10 to 60 mg per day.

34. The method of claim 1, wherein the dose of statin is at least about 10 to 50 mg per day.

35. The method of claim 1, wherein the dose of statin is at least about 10 to 40 mg per day.

36. The method of claim 1, wherein the dose of statin is at least about 20 to 40 mg per day.

37. A kit for treating a patient having multiple sclerosis, comprising a therapeutically effective dose of an agent for treating or at least partially alleviating the symptoms of multiple sclerosis, and a statin, either in the same or separate packaging, and instructions for its use.

38. The kit of claim 37, wherein said agent for treating multiple sclerosis is selected from the group consisting of β-interferons, glatiramer acetate, interferon-τ, spirogermaniums, vitamin D analogs, prostaglandins, tetracyclines, adrenocorticotrophic hormone, corticosteroid, prednisone, methylprednisone, 2-chlorodeoxyadenosine, mitoxantrone, sulphasalazine, methotrexate, azathioprine, cyclophosphamide, cyclosporin, and tizanidine hydrochloride.

39. The kit of claim 37, wherein said statin is selected from the group consisting of Compactin, Atorvastatin, Lovastatin, Mevinolin, Pravastatin, Fluvastatin, Mevastatin, visastatin/Rosuvastatin, Velostatin, Cerivastatin, Simvastatin, Synvinolin, Rivastatin (sodium 7-(4-fluorophenyl)-2,6-diisoprop-yl-5-methoxymethylpyridin-3-yl)-3,5-dihydroxy-6-heptanoate), itavastatin/pitavastatin, pharmaceutically acceptable salts and esters thereof, and combinations thereof.

40. The kit of claim 37, wherein the dose of statin is at least about 10 to 80 mg per day.

41. The kit of claim 37, wherein the dose of statin is at least about 10 to 70 mg per day.

42. The kit of claim 37, wherein the dose of statin is at least about 10 to 60 mg per day.

43. The kit of claim 37, wherein the dose of statin is at least about 10 to 50 mg per day.

44. The kit of claim 37, wherein the dose of statin is at least about 10 to 40 mg per day.

45. The kit of claim 37, wherein the dose of statin is at least about 20 to 40 mg per day.

46. A pharmaceutical composition comprising a statin and a second multiple sclerosis drug, in an effective amount to treat multiple sclerosis.

47. A pharmaceutical composition comprising a statin and a β-interferon in an effective amount to treat multiple sclerosis.

48. The composition of claim 46, wherein said second multiple sclerosis drug is selected from the group consisting of β-interferons, glatiramer acetate, interferon-τ, spirogermaniums, vitamin D analogs, prostaglandins, tetracyclines, adrenocorticotrophic hormone, corticosteroid, prednisone, methylprednisone, 2-chlorodeoxyadenosine, mitoxantrone, sulphasalazine, methotrexate, azathioprine, cyclophosphamide, cyclosporin, and tizanidine hydrochloride.

49. The composition of claim 47, wherein said interferon-β is selected from the group consisting of β-interferon is interferon-β1a, interferon-β1b, or interferon-β2.
